(19) United States
(12) Patent Application Publication
(10) Pub. No.: US 2010/0004250 A1
(43) Pub. Date: Jan. 7, 2010

(54) PTERIDINE DERIVATIVES AS POLO-LIKE KINASE INHIBITORS USEFUL IN THE TREATMENT OF CANCER

(75) Inventors: Oliver James Philips, Oxfordshire (GB); Julie Mathilde Thibaud, Oxfordshire (GB); Carl Leslie North, Oxfordshire (GB); David Festus Moffat, Oxfordshire (GB); Sanjay Ratilal Patel, Oxfordshire (GB)

Correspondence Address: BANNER & WITCOFF, LTD., 1100 13th STREET, N.W., SUITE 1200, WASHINGTON, DC, 20005-4051, US

(73) Assignee: CHROMA THERAPEUTICS LTD., OXFORDHSIRE (GB)

(21) Appl. No.: 12/447,011

(22) PCT Filed: Sep. 25, 2007
(86) PCT No.: PCT/GB07/03625

§ 371 Date: May 14, 2009

(30) Foreign Application Priority Data
Oct. 25, 2006 (GB) 0621203.9

(51) Int. Cl. A61K 31/4985; C07D 401/14; A61P 35/00; C07D 475/00

(52) U.S. Cl. 514/249; 544/258

(57) ABSTRACT

Compounds of formula (I) are inhibitors of Polo-like kinases (PLKs), and are useful, inter alia, in the treatment of proliferative diseases:

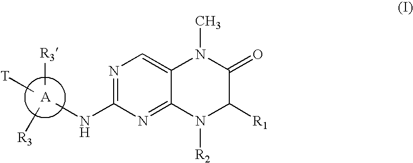

wherein R₁ is hydrogen, or a (C₁-C₆)alkyl, (C₂-C₆)alkenyl, (C₂-C₆)alkynyl or (C₃-C₆)cycloalkyl group; R₂ is hydrogen, or an optionally substituted (C₁-C₆)alkyl, (C₂-C₆)alkenyl, (C₂-C₆)alkynyl or (C₃-C₆)cycloalkyl group; R₃ and R₃′ are independently selected from hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₆R₇ or C₁-C₄ alkoxy, wherein R₆ and R₇ are independently hydrogen or optionally substituted (C₁-C₆)alkyl; ring A is an optionally substituted mono- or bi-cyclic carbocyclic or heterocyclic ring or ring system having up to 12 ring atoms; T is a radical of formula (II)

R₄R₅CH—NH—Y-L¹-X¹— (II)

Wherein R₄ is a carboxylic acid group (—COOH), or an ester group which is hydrolysable by one or more intracellular esterase enzymes to a carboxylic acid group; R₅ is the side chain of a natural or non-natural alpha amino acid; and the linker radical —Y-L¹-X¹ is as defined in the claims.

This invention relates to a series of amino acid esters, to compositions containing them, to processes for their preparation and to their use in medicine as Polo-like kinase ‘PLK’ inhibitors. Polo-like kinases (PLKs) are key enzymes that control mitotic entry of proliferating cells and regulate many aspects of mitosis necessary for successful cytokinesis. Of the four known human PLKs, PLK1 is the best characterized and is overexpressed in many tumour types with aberrant elevation frequently constituting a prognostic indicator of poor disease outcome. Accordingly, the compounds are useful in the treatment of cell proliferative diseases such as cancer. The present invention encompasses compounds that are dihydropteridinine derivatives.

### BACKGROUND TO INVENTION

The PLKs, a family of Ser/Thr protein kinases named after their functional and sequence similarity with the archetypal polo kinase from Drosophila melanogaster, play a variety of roles in mitosis (Nat. Rev. Mol. Cell. Biol., 2001, 2, 21-32.). In yeasts (Saccharomyces cerevisiae and S. pombe) single PLKs exist, whereas four distinct PLKs have been identified to date in mammals. Human PLK1 (Cell Growth Differ., 1994, 5, 249-257), PLK2 (serum-inducible kinase, SNK, Mol. Cell. Biol., 1992, 12, 4164-4169), PLK3 (proliferation-related kinase, PRK J. Biol. Chem., 1997, 272, 28646-28651) and PLK4 (Oncol. Rep., 1997, 4, 505-510) are structurally homologous and contain two conserved domains, the N-terminal catalytic kinase domain, as well as a C-terminal region composed of the so-called polo boxes. Whereas PLK1, PLK2, and PLK3 are expressed in all tissues, PLK4 appears to possess unique physiological roles and the distribution of PLK4 mRNA in adults is restricted to certain tissues such as testes and thymus.

PLK1 is the best characterized member of the PLK family and it appears to fulfil most of the known functions of the single PLKs present in invertebrates (Nat. Rev. Mol. Cell. Biol., 2004, 5, 429-441). PLK1 protein levels fluctuate in a cell-cycle-dependent manner and its kinase activity peaks at the transition between the second gap phase and the mitosis phases (G2/M) of the eukaryotic cell division cycle. Upon exit from mitosis PLK1 levels drop as a result of ubiquitin-dependent proteolysis. PLK1 has been reported to be involved in the initiation of mitosis through activation of the cyclin-dependent kinase CDK1/cyclin B complex, i.e. the master switch for mitotic entry (mitosis-promoting factor, MPF, Nature, 1990, 344, 503-508).

This occurs when PLK1 phosphorylates, and thus activates, the dual specificity phosphatase CDC25C, which in turn relieves premitotic MYT1- and WEEL-mediated suppression of CDK1/cyclin B activity through dephosphorylation at the CDK1 pThr14 and pTyr15 sites (Cell, 1991, 67, 197-211). Upon entry into mitosis, phosphorylation of CDC25C by PLK1 and PLK3 leads to its translocation into the nucleus. Apart from controlling entry into mitosis through CDK1 activation, PLK1 has additional roles in regulating progression through mitosis. It is involved in bipolar spindle formation, including centrosome maturation and regulation of the microtubule organizing centre, in the subsequent steps of mitosis involving sister chromatid separation, and finally in cytokinesis (Dev. Cell, 2003, 5, 127-138).

### BRIEF SUMMARY OF THE INVENTION

Compounds of the invention are related to compounds disclosed in WO 2004076454. They are inhibitors of PLK1 and the isoforms thereof. The compounds are thus of use in medicine, for example in the treatment of a variety of proliferative disease states, including cancers. The compounds are characterised by the presence in the molecule of an amino acid motif or an amino acid ester motif which is hydrolysable by an intracellular carboxylesterase. Compounds of the invention having the lipophilic amino acid ester motif cross the cell membrane, and are hydrolysed to the acid by the intracellular carboxylesterases. The polar hydrolysis product accumulates in the cell since it does not readily cross the cell membrane. Hence the PLK1 activity of the compound is prolonged and enhanced within the cell.

### DETAILED DESCRIPTION OF THE INVENTION

According to the invention there is provided a compound of formula (I), or a salt, N-oxide, hydrate or solvate thereof:

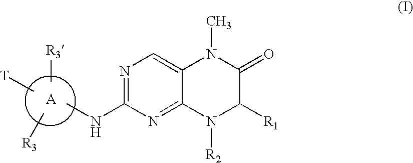

wherein
R₁ is hydrogen, or an optionally substituted (C₁-C₆)alkyl, (C₂-C₆)alkenyl, (C₂-C₆)alkynyl or (C₃-C₆)cycloalkyl group;
R₂ is hydrogen, or an optionally substituted (C₁-C₆)alkyl, (C₂-C₆)alkenyl, (C₂-C₆)alkynyl or (C₃-C₆)cycloalkyl group;
R₃ and R₃′ are independently selected from hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₆R₇ or C₁-C₄ alkoxy, wherein R₆ and R₇ are independently hydrogen or optionally substituted (C₁-C₆)alkyl;
ring A is an optionally substituted mono- or bi-cyclic carbocyclic or heterocyclic ring or ring system having up to 12 ring atoms;
T is a radical of formula (II)

R₄R₅CH—NH—Y-L¹-X¹— (II)

wherein
R₄ is a carboxylic acid group (—COOH), or an ester group which is hydrolysable by one or more intracellular esterase enzymes to a carboxylic acid group;
R₅ is the side chain of a natural or non-natural alpha amino acid;
Y is a bond, —C(═O)—, —S(═O)₂—, —C(═O)O—, —C(═O)NR₆—, —C(═S)—NR₆, —C(═NH)—NR₆ or —S(═O)₂NR₆— wherein R₆ is independently hydrogen or optionally substituted (C₁-C₆)alkyl;
L¹ is a divalent radical of formula -(Alk¹)ₘ(Q¹)ₙ(Alk²)ₚ— wherein
m, n and p are independently 0 or 1,
Q¹ is (i) an optionally substituted divalent mono- or bicyclic carbocyclic or heterocyclic radical having 5-13 ring members, or (ii), in the case where p is 0, a divalent radical of formula -Q²-X²— wherein X² is —O—, —S— or NR^{A}— wherein R^{A} is hydrogen or optionally substituted C₁-C₃ alkyl, and Q² is an optionally substituted divalent mono- or bicyclic carbocyclic or heterocyclic radical having 5-13 ring members,
Alk¹ and Alk² independently represent optionally substituted divalent (C₃-C₆)cycloalkyl radicals, or optionally substituted straight or branched, (C₁-C₆)alkylene, (C₂-C₆)alkenylene, or (C₂-C₆)alkynylene radicals which may optionally contain or terminate in an ether (—O—), thioether (—S—) or amino (—NR^{A}—) link wherein R^{A} is hydrogen or optionally substituted (C₁-C₃)alkyl;
   X¹ represents a bond, —C(═O)—; or —S(═O)₂—; —NR₆C(═O)—, —C(═O)NR₆—, —NR₆C(═O)—NR₇—, —NR₆S(═O)₂—, or —S(═O)₂NR₆— wherein R₆ and R₇ are independently hydrogen or optionally substituted (C₁-C₆)alkyl.

In the compounds of the invention, when R₁ is other than hydrogen, the carbon atom to which the R₁ substituent is attached is asymmetric. Preferably the stereochemistry at that asymmetric center is (R).

In another broad aspect the invention provides the use of a compound of formula (I) as defined above, or an N-oxide, salt, hydrate or solvate thereof in the preparation of a composition for inhibiting the activity of PLK1.

The compounds with which the invention is concerned may be used for the inhibition of PLK1 activity ex vivo or in vivo.

In one aspect of the invention, the compounds of the invention may be used in the preparation of a composition for treatment of cell proliferative diseases such as solid tumours and haemato-oncological tumours such as leukaemias and lymphomas.

In another aspect, the invention provides a method for the treatment of the foregoing disease types, which comprises administering to a subject suffering such disease an effective amount of a compound of formula (I) as defined above.

### Terminology

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to b carbon atoms, at least one double bond, and two unsatisfied valences.

As used herein the term “Cₐ-C_{b} alkynyl” wherein a and b are integers refers to straight chain or branched chain hydrocarbon groups having from a to b carbon atoms and having in addition one triple bond. For a=2 and b=6, this term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term “divalent (Cₐ-C_{b})alkynylene radical” wherein a and b are integers refers to a divalent hydrocarbon chain having from a to b carbon atoms, and at least one triple bond.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, benzfuranyl, pyranyl, isoxazolyl, benzimidazolyl, methylenedioxyphenyl, ethylenedioxyphenyl, maleimido and succinimido groups.

A “divalent phenylene, pyridinylene, pyrimidinylene, or pyrazinylene radical” is a benzene, pyridine, pyrimidine or pyrazine ring, with two unsatisfied valencies, and includes 1,3-phenylene, 1,4-phenylene, and the following:

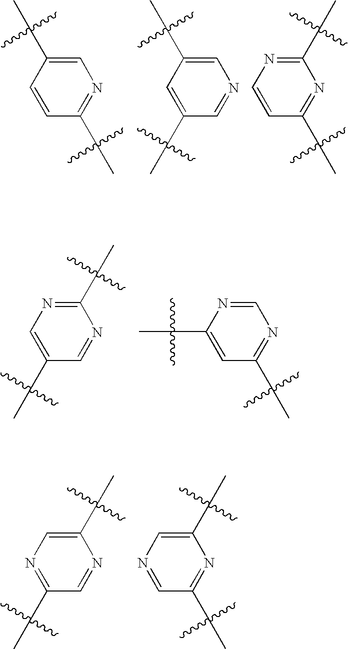

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, phenyl, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆)cycloalkyl, phenyl or monocyclic heteroaryl having 5 or 6 ring atoms, or R^{A} and R^{B} when attached to the same nitrogen atom form a cyclic amino group (for example morpholino, piperidinyl, piperazinyl, or tetrahydropyrrolyl). An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like.

Compounds of the invention which contain one or more actual or potential chiral centres, because of the presence of asymmetric carbon atoms, can exist as a number of diastereoisomers with R or S stereochemistry at each chiral centre. The invention includes all such diastereoisomers and mixtures thereof.

The term “ester” or “esterified carboxyl group” in connection with substituent R₄ above means a group R₁₀O(C═O)— in which R₁₀ is the group characterising the ester, notionally derived from the alcohol R₁₀OH.

### The Substituents R₁-R₃

R₁ is hydrogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl. In one subclass of compounds of the invention R₁ is ethyl.

R₂ is hydrogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl, or C₆₋₁₄ aryl for example phenyl or naphthyl. In one subclass of compounds of the invention R₂ is cyclopentyl.

R₃ and R₃′ are independently selected from hydrogen, —CN, hydroxyl, halogen, (C₁-C₆)alkyl, for example methyl, ethyl, n- or iso-propyl, (C₂-C₆)alkenyl, for example allyl, (C₂-C₆)alkynyl, for example —CH₂C≡CH or (C₃-C₆)cycloalkyl, for example cyclopropyl, cyclopentyl or cyclohexyl, —NR₆R₇ and (C₁-C₄)alkoxy, wherein R₆ and R₇ are independently hydrogen or optionally substituted (C₁-C₆)alkyl, for example methyl or ethyl. In one subclass of compounds of the invention R₃ is methoxy, fluoro or chloro, and R′₃ is hydrogen, fluoro or chloro.

### The Ring A

Ring A is a mono- or bi-cyclic carbocyclic or heterocyclic ring or a ring system having up to 12 ring atoms. Examples of such rings are piperidine, piperazine, pyridine, pyrimidine, pyrazoline, triazoline, furan, thophene, pyrrole, thiazole, isothiazole, oxazole, isoxazole, and thiadiazole rings. Currently preferred rings A are phenyl, pyridinyl and pyrimidinyl.

One currently preferred sub-class of compounds of the invention consists of those having formula (IA):

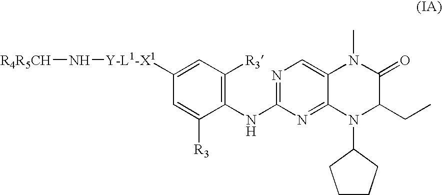

wherein R₃ is methoxy, fluoro or chloro, R′₃ is hydrogen, fluoro or chloro, and the remaining variables are as defined above and discussed further below.

### The Group R₄

R₄ is a carboxylic acid group or an ester group which is hydrolysable by one or more intracellular carboxylesterase enzymes to a carboxylic acid group. Intracellular carboxylesterase enzymes capable of hydrolysing the ester group of a compound of the invention to the corresponding acid include the three known human enzyme isotypes hCE-1, hCE-2 and hCE-3. Although these are considered to be the main enzymes, other enzymes such as biphenylhydrolase (BPH) may also have a role in hydrolysing the ester. In general, if the carboxylesterase hydrolyses the free amino acid ester to the parent acid it will also hydrolyse the ester motif when covalently conjugated to the PLK1 inhibitor. Hence, the broken cell assay described herein provides a straightforward, quick and simple first screen for esters which have the required hydrolysis profile. Ester motifs selected in that way may then be re-assayed in the same carboxylesterase assay when conjugated to the modulator via the chosen conjugation chemistry, to confirm that it is still a carboxylesterase substrate in that background.

Subject to the requirement that they be hydrolysable by intracellular carboxylesterase enzymes, examples of particular ester groups R₄ include those of formula —(C═O)OR₁₀ wherein R₁₀ is R₁₁R₁₂R₁₃C— wherein
(i) R₁₁ is hydrogen or optionally substituted (C₁-C₃)alkyl-(Z¹)ₐ-[(C₁-C₃)alkyl]_{b}—, (C₂-C₃)alkenyl-(Z¹)ₐ-[(C₁-C₃)alkyl]_{b}- or phenyl-(Z¹)ₐ-[(C₁-C₃)alkyl]_{b}-, wherein a and b are independently 0 or 1 and Z¹ is —O—, —S—, or —NR₁₄— wherein R₁₄ is hydrogen or (C₁-C₃)alkyl; and R₁₂ and R₁₃ are independently hydrogen or (C₁-C₃)alkyl-;
(ii) R₁₁ is hydrogen or optionally substituted R₁₅R₁₆N—(C₁-C₃)alkyl- wherein R₁₅ is hydrogen, (C₁-C₃)alkyl or phenyl, and R₁₆ is hydrogen or (C₁-C₃)alkyl; or R₁₅ and R₁₆ together with the nitrogen to which they are attached form an optionally substituted monocyclic heterocyclic ring of 5- or 6-ring atoms or bicyclic heterocyclic ring system of 8 to 10 ring atoms, and R₁₂ and R₁₃ are independently hydrogen or (C₁-C₃)alkyl-; or
(iii) R₁₁ and R₁₂ taken together with the carbon to which they are attached form an optionally substituted monocyclic carbocyclic ring of from 3 to 7 ring atoms or bicyclic carbocyclic ring system of 8 to 10 ring atoms, and R₁₃ is hydrogen.

Within these classes, R₄ may be, for example, a methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbonyl, dimethylaminoethyl, or morpholinoethyl ester group. Currently preferred is where R₄ is a cyclopentyl ester group.

Macrophages are known to play a key role in inflammatory disorders through the release of cytokines in particular TNFα and IL-1 (van Roon et al., Arthritis and Rheumatism, 2003, 1229-1238). In rheumatoid arthritis they are major contributors to the maintenance of joint inflammation and joint destruction. Macrophages are also involved in tumour growth and development (Naldini and Carraro, Curr Drug Targets Inflamm Allergy, 2005, 3-8). Hence agents that selectively target macrophage cell proliferation and function could be of value in the treatment of cancer and autoimmune disease. Targeting specific cell types would be expected to lead to reduced side-effects. The inventors have discovered a method of targeting inhibitors to cells that express hCE-1, in particular macrophages and other cells derived from the myelo-monocytic lineage such as monocytes, osteoclasts and dendritic cells, This is based on the observation that the way in which the esterase motif is linked to the inhibitor determines whether it is hydrolysed by all three human carboxylesterases or just by hCE-1, and hence whether or not it accumulates in different cell types. Specifically it has been found that macrophages and other cells derived from the myelo-monocytic lineage, both normal and cancerous, contain the human carboxylesterase hCE-1 whereas other cell types do not. In the general formula (I) when the nitrogen of the esterase motif R₁CH(R₂)NH— is not directly linked to a carbonyl (—C(═O)—), ie when Y is not a —C(═O), —C(═O)O— or —C(═O)NR₃— radical, the ester will only be hydrolysed by hCE-1 and hence the inhibitors selectively accumulate in macrophage-related cells

### The Amino Acid Side Chain R₅

Subject to the requirement that the ester group R₄ be hydrolysable by intracellular carboxylesterase enzymes, the identity of the side chain group R₅ is not critical.

Examples of amino acid side chains include:

(C₁-C₆)alkyl, phenyl, 2,- 3-, or 4-hydroxyphenyl, 2,- 3-, or 4-methoxyphenyl, 2-, 3-, or 4-pyridylmethyl, benzyl, phenylethyl, 2-, 3-, or 4-hydroxybenzyl, 2,- 3-, or 4-benzyloxybenzyl, 2,- 3-, or 4-(C₁-C₆)alkoxybenzyl, and benzyloxy(C₁-C₆alkyl)-groups;
the characterising group of a natural α amino acid, in which any functional group may be protected;
groups -[Alk]ₙR₁₆ where Alk is a (C₁-C₆)alkyl or (C₂-C₆)alkenyl group optionally interrupted by one or more —O—, or —S— atoms or —N(R₁₇)— groups [where R₁₇ is a hydrogen atom or a (C₁-C₆)alkyl group], n is 0 or 1, and R₁₆ is an optionally substituted cycloalkyl or cycloalkenyl group;
a benzyl group substituted in the phenyl ring by a group of formula —OCH₂COR₁₈ where R₁₈ is hydroxyl, amino, (C₁-C₆)alkoxy, phenyl(C₁-C₆)alkoxy, (C₁-C₆)alkylamino, di((C₁-C₆)alkyl)amino, phenyl(C₁-C₆)alkylamino, the residue of an amino acid or acid halide, ester or amide derivative thereof, said residue being linked via an amide bond, said amino acid being selected from glycine, α or β alanine, valine, leucine, isoleucine, phenylalanine, tyrosine, tryptophan, serine, threonine, cysteine, methionine, asparagine, glutamine, lysine, histidine, arginine, glutamic acid, and aspartic acid; a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; and a group —CRₐR_{b}R_{c} in which:
each of Rₐ, R_{b} and R_{c} is independently hydrogen, (C₁-C₆)alkyl, (C₂-C₆)alkenyl, (C₂-C₆)alkynyl, phenyl(C₁-C₆)alkyl, (C₃-C₈)cycloalkyl; or
R₁ is hydrogen and Rₐ and R_{b} are independently phenyl or heteroaryl such as pyridyl; or
R_{c} is hydrogen, (C₁-C₆)alkyl, (C₂-C₆)alkenyl, (C₂-C₆)alkynyl, phenyl(C₁-C₆)alkyl, or (C₃-C₈)cycloalkyl, and Rₐ and R_{b} together with the carbon atom to which they are attached form a 3 to 8 membered cycloalkyl or a 5- to 6-membered heterocyclic ring; or
Rₐ, R_{b} and R_{c} together with the carbon atom to which they are attached form a tricyclic ring (for example adamantyl); or
Rₐ and R_{b} are each independently (C₁-C₆)alkyl, (C₂-C₆)alkenyl, (C₂-C₆)alkynyl, phenyl(C₁-C₆)alkyl, or a group as defined for R_{c} below other than hydrogen, or Rₐ and R_{b} together with the carbon atom to which they are attached form a cycloalkyl or heterocyclic ring, and R_{c} is hydrogen, —OH, —SH, halogen, —CN, —CO₂H, (C₁-C₄)perfluoroalkyl, —CH₂OH, —CO₂(C₁-C₆)alkyl, —O(C₁-C₆)alkyl, —O(C₂-C₆)alkenyl, —S(C₁-C₆)alkyl, —SO(C₁-C₆)alkyl, —SO₂(C₁-C₆)alkyl, —S(C₂-C₆)alkenyl, —SO(C₂-C₆)alkenyl, —SO₂(C₂-C₆)alkenyl or a group -Q-W wherein Q represents a bond or —O—, —S—, —SO— or —SO₂— and W represents a phenyl, phenylalkyl, (C₃-C₈)cycloalkyl, (C₃-C₈)cycloalkylalkyl, (C₄-C₈)cycloalkenyl, (C₄-C₈)cycloalkenylalkyl, heteroaryl or heteroarylalkyl group, which group W may optionally be substituted by one or more substituents independently selected from, hydroxyl, halogen, —CN, —CO₂H, —CO₂(C₁-C₆)alkyl, —CONH₂, —CONH(C₁-C₆)alkyl, —CONH(C₁-C₆alkyl)₂, —CHO, —CH₂OH, (C₁-C₄)perfluoroalkyl, —O(C₁-C₆)alkyl, —S(C₁-C₆)alkyl, —SO(C₁-C₆)alkyl, —SO₂(C₁-C₆)alkyl, —NO₂, —NH₂, —NH(C₁-C₆)alkyl, —N((C₁-C₆)alkyl)₂, —NHCO(C₁-C₆)alkyl, (C₁-C₆)alkyl, (C₂-C₆)alkenyl, (C₂-C₆)alkynyl, (C₃-C₈)cycloalkyl, (C₄-C₈)cycloalkenyl, phenyl or benzyl.

Examples of particular R₅ groups include benzyl, phenyl, cyclohexylmethyl, cyclohexyl, pyridin-3-ylmethyl, tert-butoxymethyl, iso-butyl, sec-butyl, tert-butyl, 1-benzylthio-1-methylethyl, 1-methylthio-1-methylethyl, 1-mercapto-1-methylethyl, and phenylethyl. Presently preferred R₅ groups include phenyl, benzyl, iso-butyl, cyclohexyl and t-butoxymethyl.

For compounds of the invention which are to be administered systemically, esters with a slow rate of carboxylesterase cleavage are preferred, since they are less susceptible to pre-systemic metabolism. Their ability to reach their target tissue intact is therefore increased, and the ester can be converted inside the cells of the target tissue into the acid product.

However, for local administration, where the ester is either directly applied to the target tissue or directed there by, for example, inhalation, it will often be desirable that the ester has a rapid rate of esterase cleavage, to minimise systemic exposure and consequent unwanted side effects. In the compounds of this invention, if the carbon adjacent to the alpha carbon of the alpha amino acid ester is monosubstituted, ie R₅ is CH₂R^{z} (R^{z} being the mono-substituent) then the esters tend to be cleaved more rapidly than if that carbon is di- or tri-substituted, as in the case where R₅ is, for example, phenyl or cyclohexyl.

### The Radical —Y-L¹-X¹—

This radical (or bond) arises from the particular chemistry strategy chosen to link the amino acid ester motif R₄CH(R₅)NH— to the rest of the molecule. Clearly the chemistry strategy for that coupling may vary widely and thus many combinations of the variables Y, L¹, and X¹ are possible. However, when the inhibitor is bound to the enzyme at its active site, the ring A is located away from the enzyme, so by linking the amino acid ester motif to ring A it generally extends in a direction away from the enzyme, and thus minimises or avoids interference with the binding mode of the inhibitor. Hence the precise combination of variables making up the linking chemistry between the amino acid ester motif and the ring A will often be irrelevant to the primary binding mode of the compound as a whole. On the other hand, that linkage chemistry may in some cases pick up additional binding interactions with the enzyme, thereby enhancing binding.

It should also be noted that the benefits of the amino acid ester motif described above (facile entry into the cell, carboxylesterase hydrolysis within the cell, and accumulation within the cell of active carboxylic acid hydrolysis product) are best achieved when the linkage between the amino acid ester motif and the ring A is not a substrate for peptidase activity within the cell, which might result in cleavage of the amino acid from the molecule. Of course, stability to intracellular peptidases is easily tested by incubating the compound with disrupted cell contents, and analysing for any such cleavage.

With the foregoing general observations in mind, taking the variables making up the radical —Y-L¹-X¹— in turn:
specific preferred examples of Y are —(C═O)—, —(C═O)NH—, and —(C═O)O—. Y may also be a bond.
In the radical L¹, examples of Alk¹ and Alk² radicals, when present, include —CH₂—, —CH₂CH₂—, —CH₂CH₂CH₂—, —CH₂CH₂CH₂CH₂—, —CH═CH—, —CH═CHCH₂—, —CH₂CH═CH—, CH₂CH═CHCH₂—C≡C—, —C≡CCH₂—, CH₂C≡C—, and CH₂C≡CCH₂. Additional examples of Alk¹ and Alk² include —CH₂W—, —CH₂CH₂W—, —CH₂CH₂WCH₂—, —CH₂CH₂WCH(CH₃)—, —CH₂WCH₂CH₂—, —CH₂WCH₂CH₂WCH₂—, and —WCH₂CH₂— where W is —O—, —S—, —NH—, —N(CH₃)—, or —CH₂CH₂N(CH₂CH₂OH)CH₂—. Further examples of Alk¹ and Alk² include divalent cyclopropyl, cyclopentyl and cyclohexyl radicals.
Alk¹ and Alk² when present may also be branched chain alkyl such as —CH(CH₃)—, —C(CH₃)₂—, or in either orientation —CH₂CH(CH₃)—, —CH₂C(CH₃)₂—.
In L¹, when n is 0, the radical is a hydrocarbon chain (optionally substituted and perhaps having an ether, thioether or amino linkage). Presently it is preferred that there be no optional substituents in L¹. When both m and p are 0, L¹ is a divalent mono- or bicyclic carbocyclic or heterocyclic radical with 5-13 ring atoms (optionally substituted). When n is 1 and at least one of m and p is 1, L¹ is a divalent radical including a hydrocarbon chain or chains and a mono- or bicyclic carbocyclic or heterocyclic radical with 5-13 ring atoms (optionally substituted). When present, Q¹ may be, for example, a divalent phenyl, naphthyl, cyclopropyl, cyclopentyl, or cyclohexyl radical, or a mono-, or bi-cyclic heterocyclic radical having 5 to 13 ring members, such as piperidinyl, piperazinyl, indolyl, pyridyl, thienyl, or pyrrolyl radical.
Specifically, in some embodiments of the invention, L¹, m and p may be 0 with n being 1. In other embodiments, n and p may be 0 with m being 1. In further embodiments, m, n and p may be all 0. In still further embodiments m may be 0, n may be 1 with Q¹ being a monocyclic heterocyclic radical, and p may be 0 or 1. Alk¹ and Alk², when present, may be selected from —CH₂—, —CH₂CH₂—, and —CH₂CH₂CH₂— and Q¹ may be 1,4-phenylene.

In a some classes of compounds of the invention the radical —Y-L¹-X¹—, Y is —C(═O)—, —C(═O)O— or —C(═O)NH—; X¹ is —NHC(═O)—; and L¹ has formula (IIIA), (IIB) or (IIC):

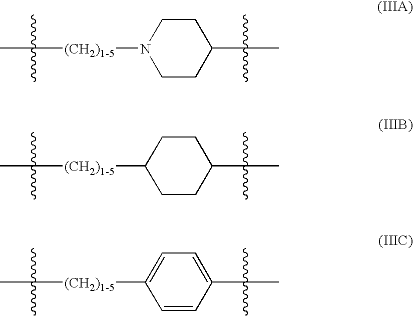

wherein the left hand valency is satisfied by Y and the right hand valency is satisfied by X¹.

Thus, a currently preferred subclass of compounds of the invention consists of those having formula (IB):

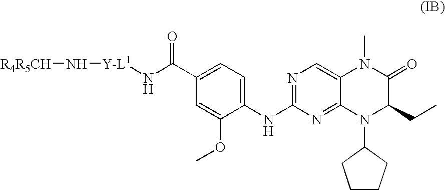

wherein R₄, R₅, Y and L¹ are as defined and more particularly discussed above.

As mentioned above, the compounds with which the invention is concerned are inhibitors of PLK1 kinase activity and are therefore of use for treatment of cell proliferative diseases such as cancer, including both solid and haemato-oncological tumours.

Specific compounds of the invention include those of the Examples herein, and the following in particular:
Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)-L-leucinate,
Cyclopentyl N-(5-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}pentanoyl)-L-leucinate
Cyclopentyl N-[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethyl)carbamoyl]-L-leucinate,
Cyclopentyl N-[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethoxy)carbonyl]-L-leucinate,
Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}butanoyl)-L-leucinate,
Cyclopentyl N-(3-{cis-4-[(4-{[(7R)-8-cyclopentyl-7-ethyl-5-methyl-6-oxo-5,6,7,8-tetrahydropteridin-2-yl]amino}-3-methoxybenzoyl)amino]cyclohexyl}propanoyl)-L-leucinate,
   and salts, hydrates and solvates thereof.

### Utilities

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application by inhalation, the drug may be formulated for aerosol delivery for example, by pressure-driven jet atomizers or ultrasonic atomizers, or preferably by propellant-driven metered aerosols or propellant-free administration of micronized powders, for example, inhalation capsules or other “dry powder” delivery systems. Excipients, such as, for example, propellants (e.g. Frigen in the case of metered aerosols), surface-active substances, emulsifiers, stabilizers, preservatives, flavorings, and fillers (e.g. lactose in the case of powder inhalers) may be present in such inhaled formulations. For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds of the invention may be used in conjunction with a number of known pharmaceutically active substances. For example, the compounds of the invention may be used with cytotoxics, HDAC inhibitors, kinase inhibitors, aminopeptidase inhibitors, protease inhibitors, bcl-2 antagonists, inhibitors of mTor and monoclonal antibodies (for example those directed at growth factor receptors). Preferred cytotoxics include, for example, taxanes, platins, anti-metabolites such as 5-fluoracil, topoisomerase inhibitors and the like. The medicaments of the invention comprising amino acid derivatives of formula (I), tautomers thereof or pharmaceutically acceptable salts, N-oxides, hydrates or solvates thereof therefore typically further comprise a cytotoxic, an HDAC inhibitor, a kinase inhibitor, an aminopeptidase inhibitor and/or a monoclonal antibody.

Hence, the present invention provides a pharmaceutical composition comprising:
(a) a compound of the invention;
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody; and
(c) a pharmaceutically acceptable carrier or diluent.

Also included is a product comprising:
(a) a compound of the invention; and
(b) a cytotoxic agent, an HDAC inhibitor, a kinase inhibitor, an aminopeptidase inhibitor, a protease inhibitor, a bcl-2 antagonist, an inhibitor of mTor and/or a monoclonal antibody,
   for the separate, simultaneous or sequential use in the treatment of the human or animal body.

### Synthesis

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to those skilled in the art. Typical literature sources are “Advanced organic chemistry”, 4^{th} Edition (Wiley), J March; “Comprehensive Organic Transformation”, 2^{nd} Edition (Wiley), R. C. Larock, “Handbook of Heterocyclic Chemistry”, 2^{nd} Edition (Pergamon), A. R. Katritzky; review articles such as found in “Synthesis”, “Acc. Chem. Res.”, “Chem. Rev”, or primary literature sources identified by standard literature searches online or from secondary sources such as “Chemical Abstracts” or “Beilstein”.

The compounds of the invention may be prepared by a number of processes some of which are described specifically in the Examples below. In the reactions described below, it may be necessary to protect reactive functional groups, for example hydroxyl, amino and carboxy groups, where these are desired in the final product, to avoid their unwanted participation in the reactions [see for example, “Protecting Groups in Organic Synthesis”, 3^{rd} Edition, (Wiley), T. W. Greene]. Conventional protecting groups may be used in conjunction with standard practice. In some instances deprotection may be the final step in the synthesis of a compound of general formula (I), and the processes according to the invention described herein after are understood to extend to such removal of protecting groups.

### Abbreviations

AcOH=acetic acid
Boc or boc=tert-butoxycarbonyl
BOC₂O=Di-tert-butyldicarbonate
Cbz=benzyloxycarbonyl
DCE=dichloroethane
DCM=dichloromethane
DIPEA=diisopropylethylamine
DMAP=dimethylamino pyridine
DMF=dimethylformamide
DMSO=dimethyl sulfoxide
EDC=1-ethyl-3-(3-dimethylaminopropyl)carbodiimide
EtOAc=ethyl acetate
EtOH=ethanol
Et₂O=diethyl ether
Et₃N=triethylamine
HCl=hydrochloric acid
HOBt=N-hydroxybenzotriazole
K₂CO₃=potassium carbonate
LiOH=lithium hydroxide
MeOH=methanol
MgSO₄=magnesium sulphate
Na₂CO₃=sodium carbonate
NaH=sodium hydride
NaHCO₃=sodium hydrogen carbonate
NaI=sodium iodide
NaOH=sodium hydroxide
NBu₄Br=tetrabutylammonium bromide
Pd(dppf)Cl₂=dichloro-(1,2-bis-(diphenylphosphino)ethane)-palladium(II)
Pd/C=palladium on carbon
PyBrOP=Bromo-tris-pyrrolidino phosphoniumhexafluorophosphate
STAB=sodium triacetoxyborohydride
TBTU=O-benzotriazol-1-yl-N,N,N′,N′-tetramethyluronium tetrafluoroborate
TFA=trifluoroacetic acid
THF=tetrahydrofuran
aq=aqueous
g=gram(s)
LCMS=high performance liquid chromatography/mass spectrometry
mg=milligram(s)
min=minutes
ml=milliliter(s)
l=microlitre(s)
mol=mole(s)
mmol=millimole(s)
NMR=nuclear magnetic resonance
RT or rt=room temperature
sat=saturated

Commercially available reagents and solvents (H PLC grade) were used without further purification. Solvents were removed using a Buchi rotary evaporator. Microwave irradiation was carried out using a Biotage initiator™, Eight microwave synthetiser. Purification of compounds by flash chromatography column was performed using silica gel, particle size 40-63 μm (230-400 mesh) obtained from Fluorochem. Purification of compounds by preparative HPLC was performed on Gilson systems using reverse phase Luna Axia™ C18 prep columns (10 μm, 100×21.2 mm), gradient 0-100% B (A=water/0.05% TFA, B=acetonitrile/0.05% TFA) over 10 min, flow=25 mL/min, UV detection at 254 nm.

¹H NMR spectra were recorded on a Bruker 300 MHz AV spectrometer in deuterated solvents. Chemical shifts (6) are in parts per million. Thin-layer chromatography (TLC) analysis was performed with Kieselgel 60 F₂₅₄ (Merck) plates and visualized using UV light.

Analytical LCMS was performed on an Agi lent HP1100 LC system using reverse phase Luna C18 columns (3 μm, 50×4.6 mm), gradient 5-95% B (A=water/0.1% Formic acid, B=acetonitrile/0.1% Formic acid) over 2.25 min, flow=2.25 mL/min. UV spectra were recorded at 220 and 254 nm using a G1315B DAD detector. Mass spectra were obtained over the range ESMS m/z: 150 to 800 on a LC/MSD SL G1956B detector. Data were integrated and reported using ChemStation and ChemStation Data Browser softwares.

### Intermediates

The intermediates for the preparation of the examples described herein are shown below (FIG. 1):

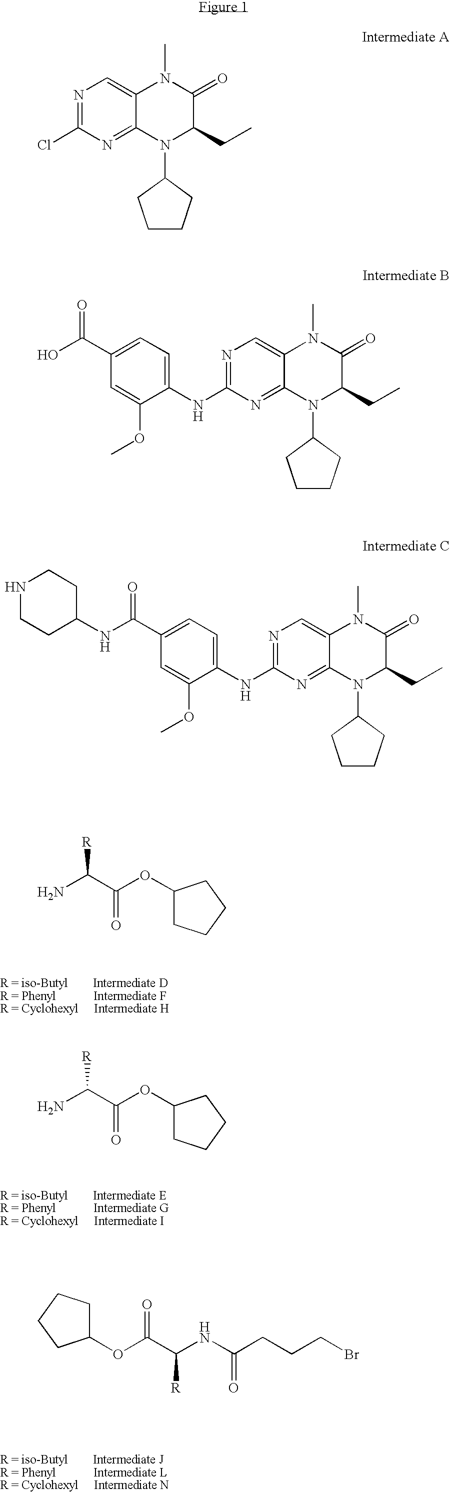

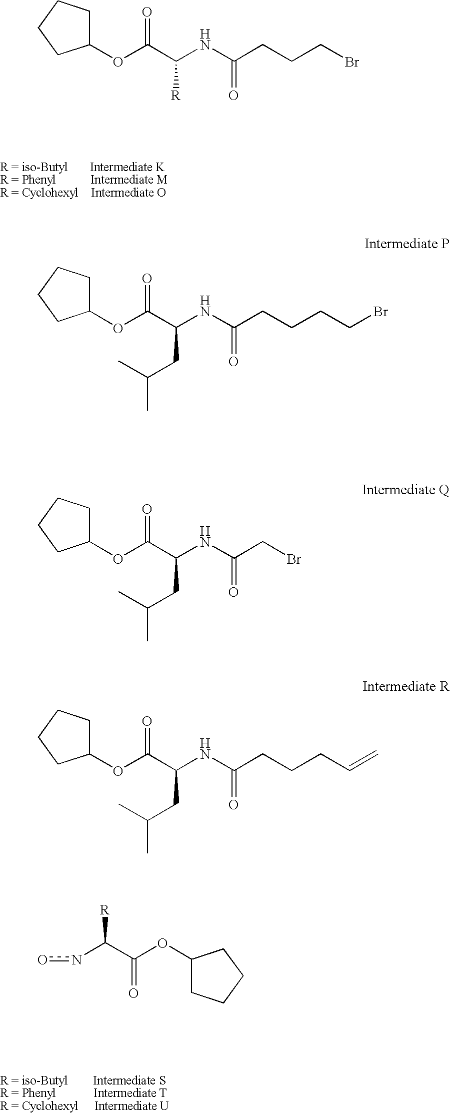

### Intermediate A

### (7R)-2-Chloro-8-cyclopentyl-7-ethyl-5-methyl-7,8-dihydropteridin-6(5H)-one

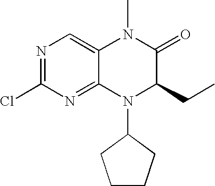

The title compound was prepared using methodology described in WO2004076454.

### Intermediate B

### 4-{[(7R)-8-Cyclopentyl-7-ethyl-5-methyl-6-oxo-5,6,7,8-tetrahydropteridin-2-yl]amino}-3-methoxybenzoic acid

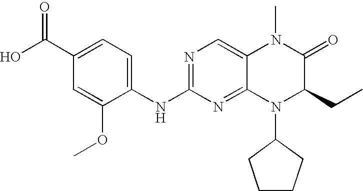

The title compound was prepared using methodology described in WO2004076454.

### Intermediate C

### 4-{[(7R)-8-Cyclopentyl-7-ethyl-5-methyl-6-oxo-5,6,7,8-tetrahydropteridin-2-yl]amino}-3-methoxy-N-piperidin-4-ylbenzamide

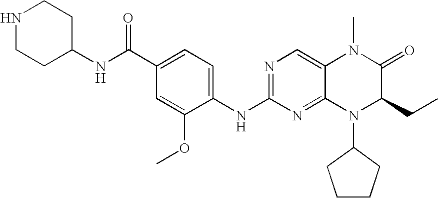

The title compound was prepared from Intermediate B by the following methodology:

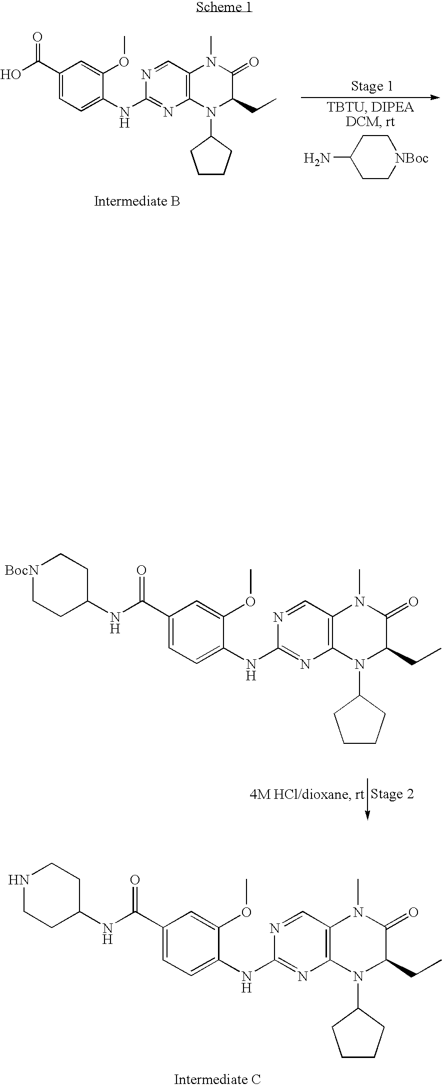

### Stage 1—tert-Butyl 4-[(4-{[(7R)-8-cyclopentyl-7-ethyl-5-methyl-6-oxo-5,6,7,8-tetrahydropteridin-2-yl]amino}-3-methoxybenzoyl)amino]piperidine-1-carboxylate

To a suspension of Intermediate B (500 mg, 1.18 mmol) in DCM (20 ml) was added TBTU (415 mg, 1.29 mmol) and DIPEA (0.41 ml, 2.35 mmol). The reaction mixture was stirred at RT for 30 min and tert-butyl 4-aminopiperidine-1-carboxylate (282 mg, 1.41 mmol) was added. The reaction mixture was stirred at RT for 30 min, diluted with DCM (30 ml), washed with water (2×30 ml), dried (MgSO₄), and concentrated under reduced pressure to leave a thick brown oil. Trituration with Et₂O/heptane (1:3) afforded the title product as a beige solid (528 mg, 74%). ESMS m/z: 608 [M+H]⁺.

### Stage 2—4-{[(7R)-8-Cyclopentyl-7-ethyl-5-methyl-6-oxo-5,6,7,8-tetrahydropteridin-2-yl]amino}-3-methoxy-N-piperidin-4-ylbenzamide (Intermediate C)

Stage 1 product (528 mg, 0.87 mmol) was suspended in a 4M HCl in dioxane (10 ml) and the reaction mixture was stirred at RT for 1 hour and then concentrated under reduced pressure. The residue was triturated with Et₂O and then partitioned between DCM (100 ml) and sat Na₂CO₃ (50 ml). The organic layer was separated, washed with sat Na₂CO₃ (50 ml), dried (MgSO₄) and concentrated under reduced pressure to afford the title intermediate as a thick yellow oil, which solidified on standing (407 mg, 92%). ESMS m/z 508 [M+H]⁺. ¹H NMR (300 MHz, CDCl₃) δ: 8.55 (1H, d, J=8.4 Hz), 7.70 (1H, s), 7.60 (1H, s), 7.44 (1H, d, J=1.8 Hz), 7.25 (1H, dd, J=2.1, 8.7 Hz), 5.95 (1H, d, J=7.8 Hz), 4.53 (1H, t, J=7.8 Hz), 4.23 (1H, dd, J=3.6, 7.8 Hz), 3.95 (3H, s), 3.34 (3H, s), 3.12 (2H, dt, J=3.0, 9.3 Hz), 2.82 (4H, s), 2.05 (4H, m), 1.75 (10H, m), 1.47 (2H, m), 0.90 (3H, t, J=7.5 Hz).

### Intermediate D

### Cyclopentyl L-Leucinate Hydrochloride

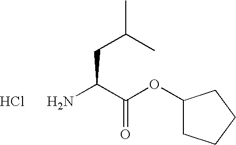

The title compound was prepared by the following methodology (Scheme 2):

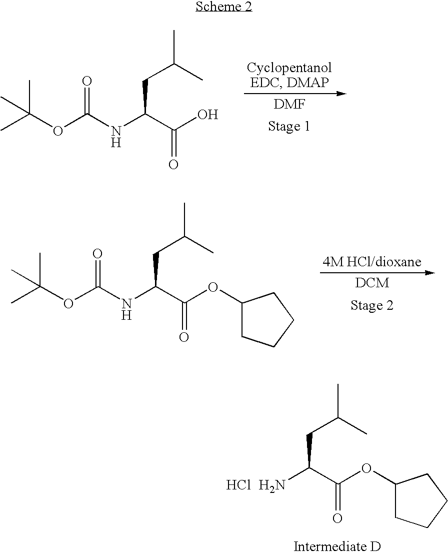

### Stage 1—Cyclopentyl N-(tert-butoxycarbonyl)-L-leucinate

To a solution of N-(tert-butoxycarbonyl)-L-leucine (22.7 g, 98.2 mmol) in anhydrous DMF (200 ml) was added cyclopentanol (17.8 ml, 0.20 mol), EDC (20.7 g, 0.11 mol) and DMAP (1.20 g, 9.82 mmol). The reaction mixture was stirred at RT for 16 hours, concentrated under reduced pressure and partitioned between water (200 ml) and EtOAc (200 ml). The aqueous layer was extracted with EtOAc (200 ml). The combined organic layers were dried (MgSO₄) and concentrated under reduced pressure. The residue was purified by column chromatography (0-30% EtOAc in heptane) to afford the title product as a colourless oil (18.01 g, 61%).

### Stage 2—Cyclopentyl L-leucinate hydrochloride (Intermediate D)

Stage 1 product (18.01 g, 60.2 mmol) was dissolved in DCM (200 ml) and 4M HCl in dioxane (30.1 ml, 0.12 mol) was added. The reaction was incomplete after stirring at RT for 72 hours, and further 4M HCl in dioxane (15 ml, 60.2 mmol) was added. The reaction was stirred for 6 hours, concentrated under reduced pressure to afford the title intermediate as a white solid (13.0 g, 92%). ESMS m/z: 200 [M+H]⁺; ¹H NMR (300 MHz, CDCl₃) δ: 5.11-5.22 (1H, m), 3.38 (1H, dd, J=8.4, 5.9 Hz), 1.23-1.94 (11H, m), 0.90 (6H, t, J=6.4 Hz).

### Intermediate E

### Cyclopentyl D-Leucinate Hydrochloride

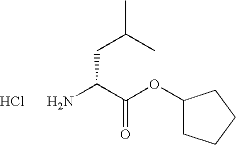

The title compound was prepared from N-(tert-Butoxycarbonyl)-D-leucine using the same methodology described for Intermediate D. ¹H NMR (300 MHz, d6-DMSO) δ: 8.20-8.35 (2H, br s), 5.10-5.20 (1H, m), 3.90 (1H, t, J=7.2 Hz), 1.50-1.95 (11H, m), 0.90 (6H, dd, J=6.6, 2.1 Hz).

### Intermediate F

### Cyclopentyl (2S)-amino(phenyl)acetate 4-methylbenzenesulfonate

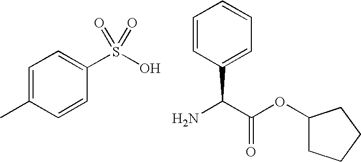

The title compound was prepared by the following methodology (Scheme 3):

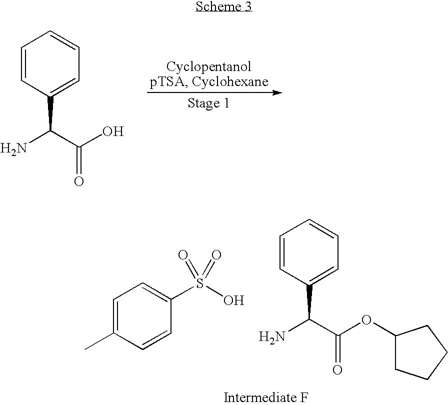

### Stage 1—Cyclopentyl (2S)-amino(phenyl)acetate tosylate salt (Intermediate F)

To a slurry of (S)-phenylglycine (5 g, 33.1 mmol) in cyclohexane (150 ml) was added cyclopentanol (29.8 ml, 0.33 mol) and p-toluene sulfonic acid (6.92 g, 36.4 mmol). The reaction was fitted with a Dean-Stark receiver and heated to 135° C. for complete dissolution. After 12 hours, the reaction was cooled to RT leading to the precipitation of a white solid. The solid was filtered and washed with EtOAc before drying under reduced pressure to give the title intermediate as a white powder (11.0 g, 85%). ¹H NMR (300 MHz, d₆-DMSO) δ: 8.82 (2H, br s), 8.73 (1H, br s), 7.47 (7H, m), 7.11 (2H, d), 5.25 (1H, br s), 5.18 (1H, m), 2.29 (3H, s), 1.87-1.36 (8H, m).

### Intermediate G

### Cyclopentyl (2R)-amino(phenyl)acetate 4-methyl benzenesulfonate

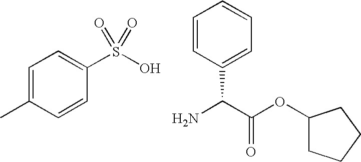

The title compound was prepared from (R)-phenylglycine using the same methodology described for Intermediate F. ¹H NMR (300 MHz, d₆-DMSO) δ: 8.80 (2H, br s), 8.74 (1H, br s), 7.44 (7H, m), 7.13 (2H, d), 5.28 (1H, br s), 5.21 (1H, m), 2.26 (3H, s), 1.85-1.30 (8H, m).

### Intermediate H

### Cyclopentyl (2S)-amino(cyclohexyl)acetate 4-methylbenzenesulfonate

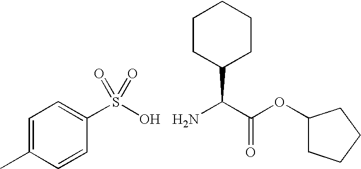

The title compound was prepared from (2S)-amino(cyclohexyl)acetic acid using the same methodology described for Intermediate F. ¹H NMR (300 MHz, d₆-DMSO) δ: 8.45 (3H, br s), 5.22 (1H, t), 3.28 (1H, d), 1.95-1.50 (10H, br m), 1.30-0.90 (9H, br m).

### Intermediate I

### Cyclopentyl (2R)-amino(cyclohexyl)acetate 4-methylbenzenesulfonate

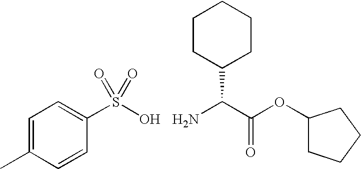

The title compound was prepared from (2R)-amino(cyclohexyl)acetic acid using the same methodology described for Intermediate F. ¹H NMR (300 MHz, CDCl₃) δ: 7.89 (3H, br s), 5.14 (1H, m), 3.79 (1H, d), 1.79-1.58 (10H, br m), 1.21-0.92 (9H, br m).

Intermediates D to I were used in aminoacid coupling reactions as free bases. To an individual skilled in the art, it will be apparent that each free base can be prepared prepared by titration of the salts described above with a suitable inorganic base (e.g. aqueous NaHCO₃).

### Intermediate J

### Cyclopentyl N-(4-bromobutanoyl)-L-leucinate

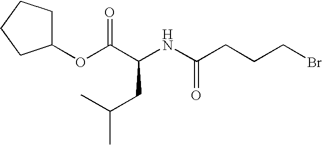

The title compound was prepared by the following methodology (Scheme 4):

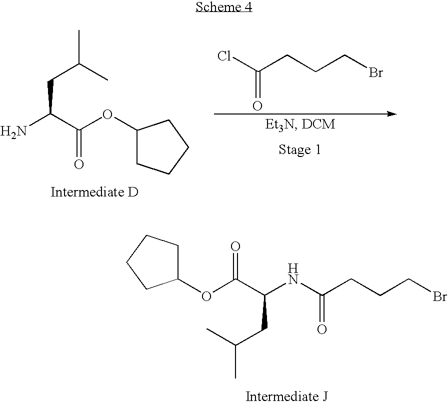

### Stage 1—Cyclopentyl N-(4-bromobutanoyl)-L-leucinate

To a solution of Intermediate D (free base, 1.82 g, 9.1 mmol) in DCM (25 ml) at 0° C. was added Et₃N (1.40 ml, 1.0 mmol) and 4-bromobutyryl chloride (1.10 ml, 9.1 mmol). The reaction mixture was allowed to warm to RT and stirred for a further 18 hours. The mixture was diluted with DCM, washed with water (2×20 ml), brine (2×20 ml), dried (MgSO₄), and concentrated under reduced pressure. Purification by column chromatography (10-30% EtOAc in heptane) afforded the title intermediate as a pale yellow oil (2.97 g, 93%). ¹H NMR (300 MHz, CDCl₃) δ: 5.91 (1H, br d, J=7.8 Hz), 5.24-5.20 (1H, m), 4.62-4.55 (1H, m), 3.65-3.59 (1H, m), 3.54-3.47 (1H, m), 2.43 (2H, t, J=7.1 Hz), 2.25-2.09 (2H, m), 1.92-1.55 (10H, m), 1.01-0.93 (7H, m).

### Intermediate K

### Cyclopentyl N-(4-bromobutanoyl)-D-leucinate

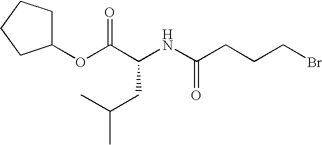

The title compound was prepared from Intermediate E using the same methodology described for Intermediate J. ESMS m/z: 349 [M+H]⁺; ¹H NMR (300 MHz, CDCl₃) δ: 5.91 (1H, br d, J=7.8 Hz), 5.24-5.20 (1H, m), 4.62-4.55 (1H, m), 3.65-3.59 (1H, m), 3.54-3.47 (1H, m), 2.43 (2H, t, J=7.1 Hz), 2.25-2.09 (2H, m), 1.92-1.55 (10H, m), 1.01-0.93 (7H, m).

### Intermediate L

### Cyclopentyl (2S)-[(4-bromobutanoyl)amino](phenyl)acetate

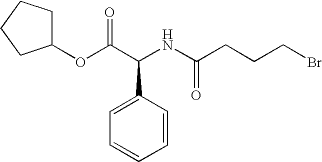

The title compound was prepared from Intermediate F using the same methodology described for Intermediate J. ESMS m/z: 368 [M+H]⁺; ¹H NMR (300 MHz, d₆-DMSO) δ: 8.81 (1H, br s), 7.50-7.38 (5H, m), 5.32-5.29 (1H, m), 5.10-5.02 (1H, m), 3.55-3.46 (2H, m), 2.50-2.32 (4H, m), 2.14-1.39 (8H, m).

### Intermediate M

### Cyclopentyl (2R)-[(4-bromobutanoyl)amino](phenyl)acetate

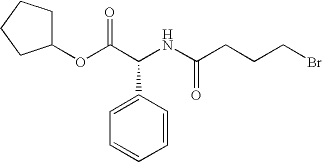

The title compound was prepared from Intermediate G using the same methodology described for Intermediate J. ESMS m/z: 368 [M+H]⁺. ¹H NMR (300 MHz, d₆-DMSO) δ: 8.81 (1H, br s), 7.50-7.38 (5H, m), 5.32-5.29 (1H, m), 5.10-5.02 (1H, m), 3.55-3.46 (2H, m), 2.50-2.32 (4H, m), 2.14-1.39 (8H, m).

### Intermediate N

### Cyclopentyl (2S)-[(4-bromobutanoyl)amino](cyclohexyl)acetate

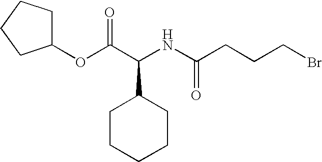

The title compound was prepared from Intermediate H using the same methodology described for Intermediate J. ESMS m/z: 374 [M+H]⁺. ¹H NMR (300 MHz, CDCl₃) δ: 5.16-5.12 (1H, m), 5.24-5.20 (1H, m), 4.43 (1H, dd J=5.0, 8.7 Hz), 3.56-3.47 (2H, m), 2.51-2.38 (2H, m), 2.39-2.33 (2H, m), 1.79-1.50 (11H, m), 1.21-0.85 (8H, m).

### Intermediate O

### Cyclopentyl (2R)-[(4-bromobutanoyl)amino](cyclohexyl)acetate

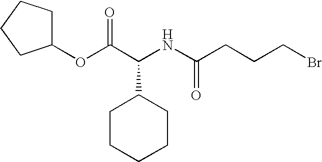

The title compound was prepared from Intermediate I using the same methodology described for Intermediate J. ESMS m/z: 374 [M+H]⁺; ¹H NMR (300 MHz, CDCl₃) δ: 5.16-5.12 (1H, m), 5.24-5.20 (1H, m), 4.43 (1H, dd J=5.0, 8.7 Hz), 3.56-3.47 (2H, m), 2.51-2.38 (2H, m), 2.39-2.33 (2H, m), 1.79-1.50 (11H, m), 1.21-0.85 (8H, m).

### Intermediate P

### Cyclopentyl N-(5-bromopentanoyl)-L-leucinate

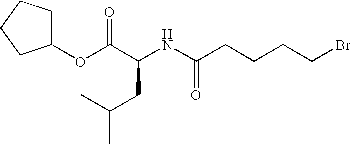

The title compound was prepared from Intermediate D and 5-bromopentanoyl chloride using the same methodology described for Intermediate J. ESMS m/z: 362 [M+H]⁺. ¹H NMR (300 MHz, CDCl3) δ: 5.88 (1H, d, J=7.5 Hz), 5.19 (1H, m), 4.55 (1H, m), 3.40 (2H, t, J=3.3 Hz), 2.24 (2H, t, J=6.9 Hz), 1.70 (14H, m), 0.95 (6H, d, J=6.3 Hz).

### Intermediate Q

### Cyclopentyl N-(bromoacetyl)-L-leucinate

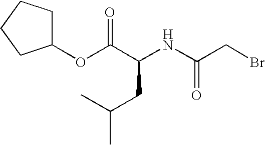

The title compound was prepared from Intermediate D and bromoacetyl chloride using the same methodology described for Intermediate J. ES MS m/z 320 [M+H]⁺. ¹H NMR (300 MHz, CDCl3) δ: 5.88 (1H, d, J=7.5 Hz), 5.19 (1H, m), 4.55 (1H, m), 3.40 (2H, t, J=3.3 Hz), 2.24 (2H, t, J=6.9 Hz), 1.70 (14H, m), 0.95 (6H, d, J=6.3 Hz).

### Intermediate R

### Cyclopentyl N-hex-5-enoyl-L-leucinate

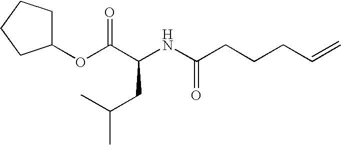

The title compound was prepared by the following methodology (Scheme 5):

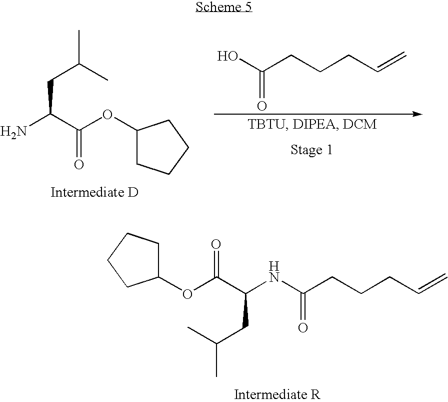

### Stage 1—Cyclopentyl N-hex-5-enoyl-L-leucinate

To a solution of 5-hexenoic acid (250 mg, 2.19 mmol) in DCM (3 ml) was added DiPEA (0.76 ml, 4.38 mmol) and TBTU (774 mg, 2.41 mmol). The mixture was stirred for 30 min before Intermediate D (524 mg, 2.63 mmol) in DCM (1 ml) was added dropwise. After stirring at RT for 1 h, the reaction mixture was diluted with DCM (10 ml) and washed with water (2 ml) and brine (2 ml). The organic layer was dried (MgSO₄), and concentrated under reduced pressure. The crude product was purified by column chromatography (16% EtOAc in heptane) to afford the title intermediate as a colourless oil in quantitative yield. ¹H NMR (300 MHz, CD3OD) δ: 5.89 (1H, d, J=8.1 Hz), 5.65-5.87 (1H, m), 5.20 (1H, dd, J=8.3, 3.2 Hz), 5.06 (1H, q, J=1.6 Hz), 4.92-5.03 (2H, m), 4.59 (1H, td, J=8.5, 5.5 Hz), 2.22 (2H, t, J=7.4 Hz), 2.10 (2H, q, J=7.0 Hz), 1.56-1.90 (12H, m), 0.95 (6H, d, J=5.7 Hz)

### Intermediate S

### Cyclopentyl N-(oxomethylene)-L-leucinate

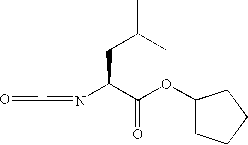

The title compound was prepared by the following methodology:

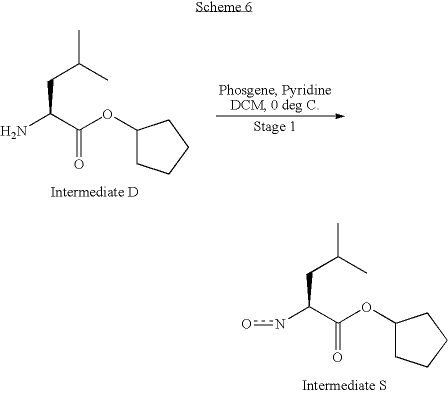

### Stage 1—Cyclopentyl N-(oxomethylene)-L-leucinate

To a solution of Intermediate D (free base, 1 g, 5.0 mmol) in DCM (15 ml) at 0° C. was added pyridine (1.6 ml, 20 mmol) followed by dropwise addition of phosgene (20% in toluene, 3.2 ml, 6.5 mmol). The reaction mixture was stirred for 2 h at 0° C., diluted with DCM (15 ml), washed with ice cold 5% aqueous HCl (2×15 ml) and cold brine (2×15 ml) dried (MgSO₄), and concentrated under reduced pressure to afford the title intermediate. It was used in the next stage without any purification.

### Intermediate T

### Cyclopentyl (2S)-isocyanato(phenyl)acetate

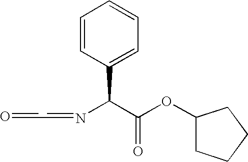

The title compound was prepared from Intermediate F using the same methodology described for Intermediate S. ¹H NMR (300 MHz, CDCl3) δ: 7.33 (5H, m), 5.25 (1H, m), 5.00 (1H, s), 1.80 (4H, m), 1.56 (4H, m).

### Intermediate U

### Cyclopentyl (2S)-cyclohexyl(isocyanato)acetate

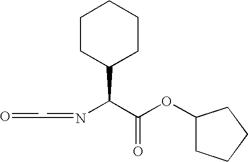

The title compound was prepared from Intermediate H using the same methodology described for Intermediate S. ¹H NMR (300 MHz, CDCl3) δ: 5.29 (1H, m), 5.09 (1H, s), 3.84 (1H, d, J=3.9 Hz), 1.77 (14H, m), 1.15 (4H, m).

### EXAMPLES

The following are representative examples of the compounds claimed by the invention

### Example 1

### Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)-L-leucinate

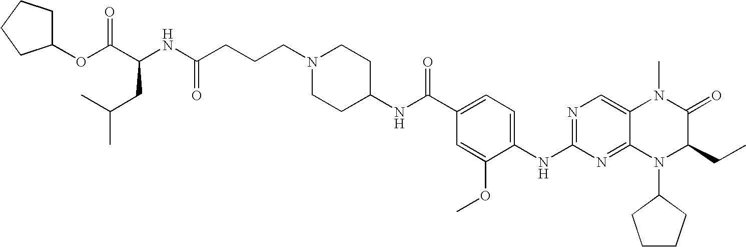

The title compound was prepared by the following methodology (Scheme 7):

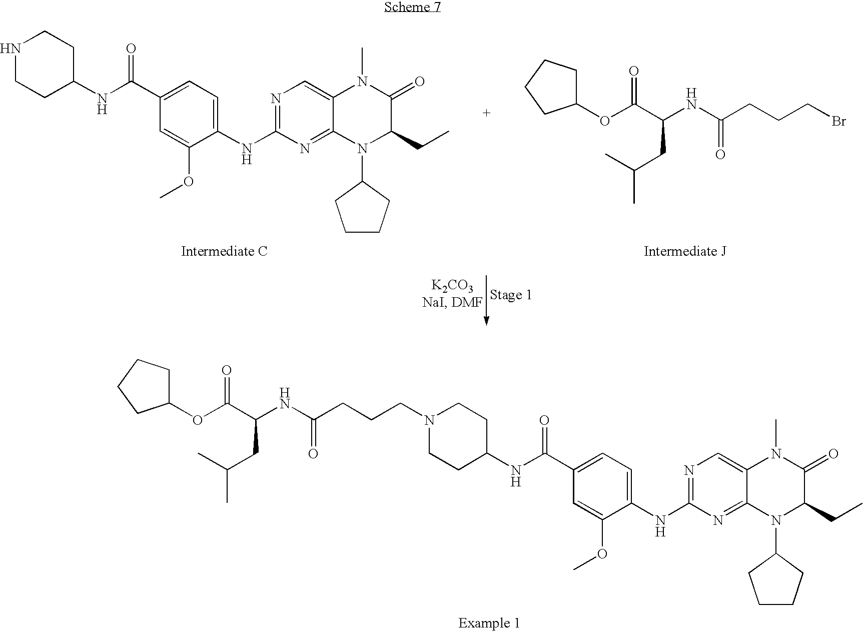

### Stage 1—Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)-L-leucinate (Example 1)

To a solution of Intermediate C (80 mg, 0.16 mmol) in DMF (2 ml) were added Intermediate J (82 mg, 0.24 mmol), K₂CO₃ (87 mg, 0.63 mmol) and NaI (47 mg, 0.32 mmol). The reaction mixture was stirred for 18 hours at 80° C. An additional equivalent of Intermediate J (55 mg, 0.16 mmol) was added. The reaction mixture was stirred for an additional 2 hours at 80° C., allowed to cool to RT and diluted with EtOAc (20 ml). The mixture washed with water (2×20 ml), brine (20 ml), dried (MgSO₄), and concentrated under reduced pressure to give a yellow oil. Purification by column chromatography (10% MeOH in DCM) afforded the title compound as a white solid (55 mg, 45%). LCMS purity 99%; ESMS m/z: 775 [M+H]⁺; ¹H NMR (300 MHz, CD₃OD) δ: 8.49 (1H, d, J=9.0 Hz), 7.77 (1H, s), 7.51-7.48 (2H, m), 5.20-5.15 (1H, m), 4.54-4.48 (1H, m), 4.38 (1H, t, J=7.5 Hz), 4.28 (1H, dd, J=3.6, 7.5 Hz), 4.01 (3H, s), 3.97-3.90 (1H, m), 3.31 (3H, s), 3.09-3.05 (2H, m), 2.50 (2H, t, J=7.7 Hz), 2.33-2.14 (5H, m), 2.01-1.58 (26H, m), 0.99 (3H, d, J=6.3 Hz), 0.94 (3H, d, J=6.3 Hz), 0.87 (3H, q, J=7.5 Hz).

### Example 2

### Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)-D-leucinate

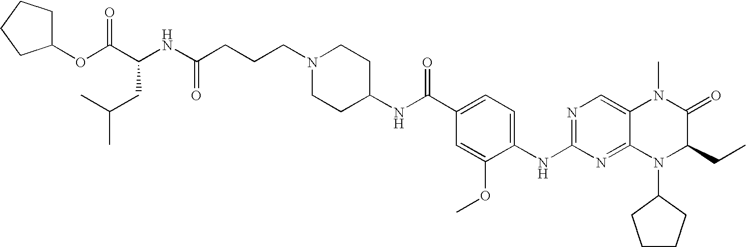

The title compound was prepared from Intermediate C and Intermediate K using the same methodology described for Example 1. LCMS purity 92%; ESMS m/z: 775 [M+H]⁺; ¹H NMR (300 MHz, CD₃OD) δ: 8.51 (1H, d, J=9.0 Hz), 7.79 (1H, s), 7.51-7.49 (2H, m), 5.18 (1H, m), 4.54 (1H, m), 4.38 (1H, t J=7.5 Hz), 4.30 (1H, dd J=3.7, 7.6 Hz), 4.02 (3H, s), 3.90 (1H, m) 3.34 (3H, s), 3.09 (2H, m), 2.52 (2H, m), 2.31-1.52 (3H, m), 0.98 (3H, d J=6.5 Hz), 0.94 (3H, d, J=6.4 Hz) 0.88 (3H, t, J=7.5 Hz).

### Example 3

### Cyclopentyl (2S)-[(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)amino](Phenyl)acetate

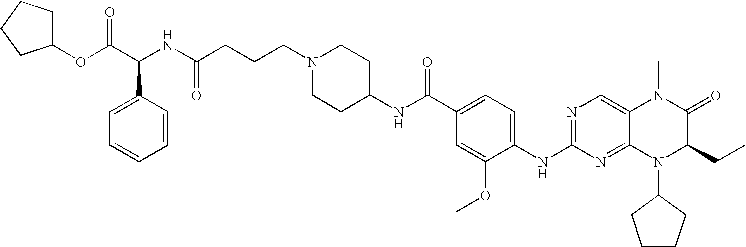

The title compound was prepared from Intermediate C and Intermediate L using the same methodology described for Example 1. LCMS purity 92%; ESMS m/z: 795 [M+H]⁺; ¹H NMR (300 MHz, CD₃OD) δ: 8.51 (1H, d, J=9.1 Hz), 7.80 (1H, s), 7.51-7.48 (2H, m), 7.40-7.38 (5H, m), 5.41 (1H, s), 5.21-5.17 (1H, m), 4.57-4.51 (1H, m), 4.32-4.28 (1H, m), 4.02 (3H, s), 3.93-3.89 (1H, m) 3.31 (3H, s), 3.09-3.04 (2H, m), 2.52-2.47 (2H, m), 2.38-2.14 (5H, m), 2.01-1.50 (23H, m), 0.87 (3H, t, J=7.4 Hz).

### Example 4

### Cyclopentyl (2R)-[(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)amino](phenyl)acetate

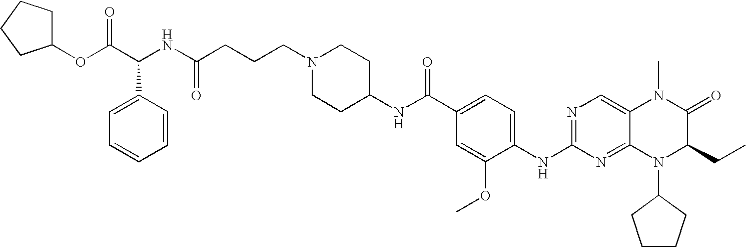

The title compound was prepared from Intermediate C and Intermediate M using the same methodology as described for Example 1. LCMS purity 93%; ESMS m/z: 795 [M+H]⁺; ¹H NMR (300 MHz, CD₃OD) δ: 8.51 (1H, d, J=7.9 Hz), 7.80 (1H, s), 7.53-7.49 (2H, m), 7.40-7.35 (5H, m), 5.41 (1H, s), 5.21-5.18 (1H, m), 4.54 (1H, m), 4.30 (1H, dd J=3.6, 7.5 Hz), 4.02 (3H, s), 3.95-3.90 (1H, m) 3.37 (3H, s), 3.12 (2H, m), 2.86 (2H, m), 2.39-2.16 (5H, m), 2.04-1.52 (22H, m), 0.88 (3H, t, J=7.4 Hz).

### Example 5

### Cyclopentyl (2R)-cyclohexyl [(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)amino]acetate

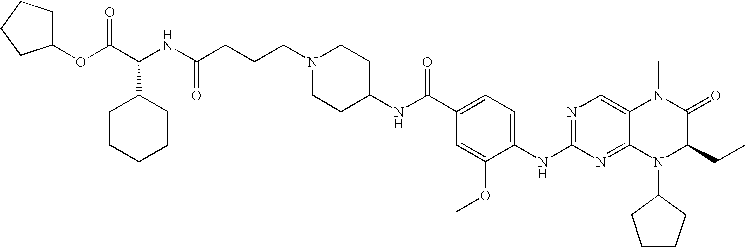

The title compound was prepared from Intermediate C and Intermediate O using the same methodology described for Example 1. LCMS purity 90%; ESMS m/z: 801 [M+H]⁺; ¹H NMR (300 MHz, CD₃OD) δ: 8.60 (1H, d, J=9.4 Hz), 7.92 (1H, d, J=7.4 Hz), 7.64 (2H, m), 5.20 (1H, m), 4.54 (1H, m), 4.40-4.16 (2H, m), 4.00 (3H, s), 3.74-3.69 (1H, m) 3.34 (3H, s), 3.25-3.09 (2H, m), 2.52 (2H, m), 2.27-1.70 (34H, m), 1.40-1.15 (5H, m), 0.88 (3H, t, J=7.5 Hz).

### Example 6

### Cyclopentyl N-(5-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}pentanoyl)-L-leucinate

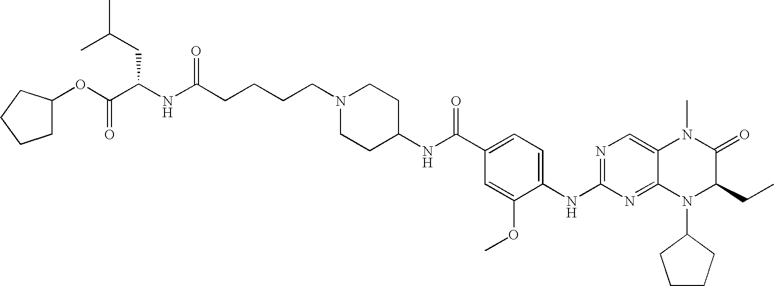

The title compound was prepared from Intermediate C and Intermediate P using the same methodology described for Example 1. LCMS purity 99%; ESMS m/z: 789 [M+H]⁺; ¹H NMR (300 MHz, CDCl₃) δ: 8.47 (1H, d, J=8.5 Hz), 7.61 (1H, s), 7.52 (1H, s), 7.35 (1H, d, J=1.7 Hz), 7.13-7.19 (1H, m), 5.92 (2H, t, J=9.1 Hz), 5.07-5.17 (1H, m), 4.36-4.57 (2H, m), 4.15 (1H, dd, J=7.9, 3.8 Hz), 3.93-4.04 (1H, m), 3.91 (3H, s), 3.26 (3H, s), 2.81-2.98 (2H, m), 2.37 (2H, t, J=7.1 Hz), 2.19 (4H, t, J=7.3 Hz), 1.89-2.05 (4H, m), 1.71-1.83 (6H, m), 1.55-1.68 (14H, m), 0.87 (6H, d, J=6.2 Hz), 0.81 (3H, t, J=7.5 Hz).

### Example 7

### Cyclopentyl N-({4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}acetyl)-L-leucinate

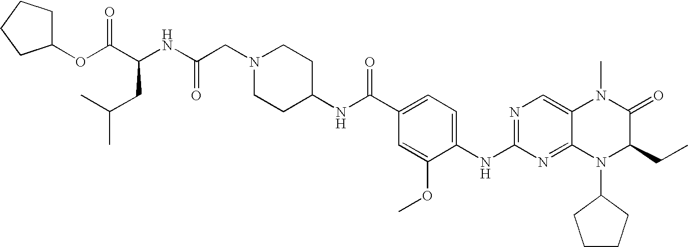

The title compound was prepared from Intermediate C and Intermediate Q using the same methodology described for Example 1. LCMS purity 97%; ESMS m/z: 747 [M+H]⁺; ¹H NMR (300 MHz, CDCl₃) δ: 8.55 (1H, d, J=8.3 Hz), 7.69 (2H, s), 7.43 (1H, d, J=1.5 Hz), 7.18-7.25 (1H, m), 5.90-6.04 (1H, m), 5.21 (1H, t, J=5.7 Hz), 4.47-4.67 (2H, m), 4.24 (1H, dd, J=7.8, 3.7 Hz), 3.99 (3H, s), 3.34 (3H, s), 2.83-3.14 (4H, m), 1.98-2.21 (4H, m), 1.52-1.92 (23H, m), 0.97 (6H, d, J=6.0 Hz), 0.89 (3H, t, J=7.4 Hz).

### Example 8

### Cyclopentyl N-[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethyl)carbamoyl]-L-leucinate

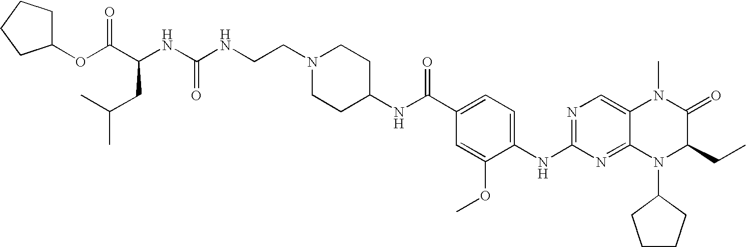

The title compound was prepared by the following methodology (Scheme 8):

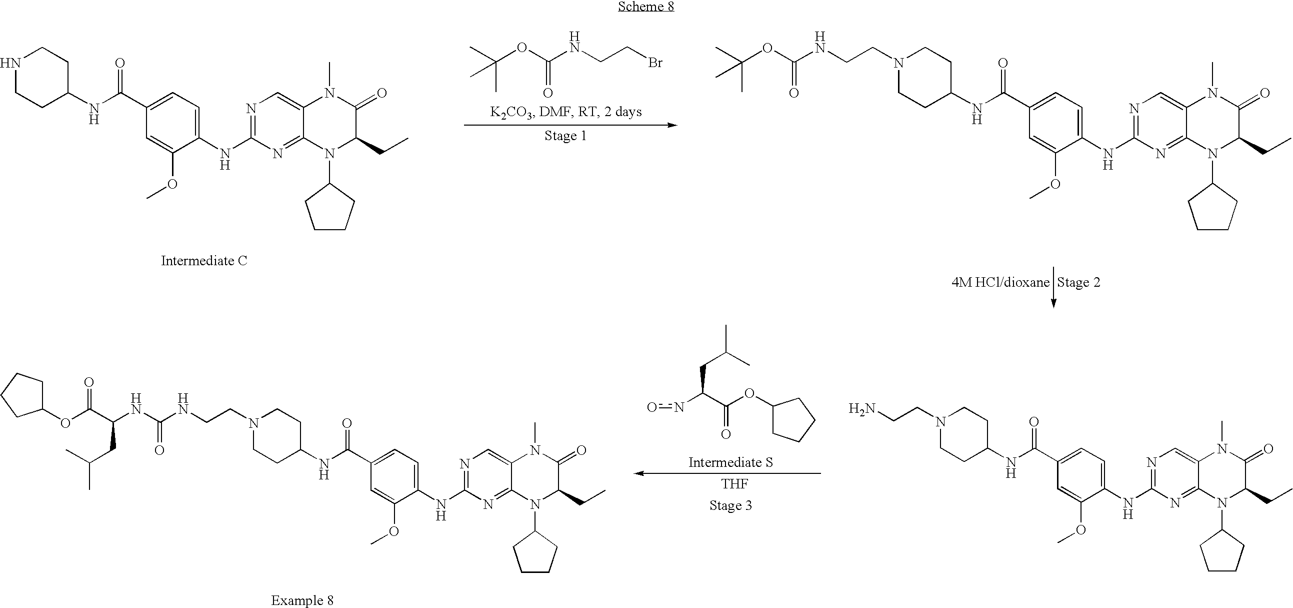

### Stage 1—tert-Butyl (2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethyl)carbamate

To a solution of Intermediate C (150 mg, 0.29 mmol) in DMF (3 ml) was added K₂CO₃ (82 mg, 0.59 mmol) and N-Boc-2-aminoethylbromide (66 mg, 0.29 mmol). The solution was stirred at RT for 96 hours and then partitioned between water (10 ml) and EtOAc (10 ml). The aqueous layer was extracted again with EtOAc 2×10 ml) and the combined organic layers were washed with water (15 ml) and brine (15 ml), dried (MgSO₄), and concentrated under reduced pressure. Purification by column chromatography (5% MeOH in DCM) afforded the title product (130 mg, 66%). ESMS m/z: 651 [M+H]⁺.

### Stage 2—N-[1-(2-Aminoethyl)piperidin-4-yl]-4-{[(7R)-8-cyclopentyl-7-ethyl-5-methyl-6-oxo-5,6,7,8-tetrahydropteridin-2-yl]amino}-3-methoxybenzamide

Stage 1 product (130 mg, 0.20 mmol) was dissolved in DCM (1 ml) and 4M HCl in dioxane (0.10 ml, 0.4 mmol) was added. The reaction mixture was stirred at RT for 1 h and solvent removed under reduced pressure. The crude residue was taken up in DCM (5 ml), washed with saturated aqueous Na₂CO₃, dried (MgSO₄), and concentrated under reduced pressure to afford the title compound (85 mg, 77%). ESMS m/z: 551 [M+H]⁺.

### Stage 3—Cyclopentyl N-[(2-{4-[(4-{[(7R)-8-cyclopentyl-7-ethyl-5-methyl-6-oxo-5,6,7,8-tetrahydro pteridin-2-yl]amino}-3-methoxybenzoyl)amino]piperidin-1-yl}ethyl)carbamoyl]-L-leucinate (Example 8)

Stage 2 product (42 mg, 0.08 mmol) was dissolved in THF (1 ml) and Intermediate S (17 mg, 0.08 mmol) was added. The reaction mixture was stirred at RT for 18 hours and the solvent removed under reduced pressure. Purification by column chromatography (5% aq. NH₃ and 5% MeOH in DCM) afforded the title compound (25 mg, 42%). ESMS m/z: 388 [(M+2H)/2]⁺; ¹H NMR (300 MHz, CDCl₃) δ: 8.49 (1H, d, J=8.5 Hz), 7.61 (1H, s), 7.53 (1H, s), 7.38 (1H, d, J=1.5 Hz), 7.24 (1H, dd, J=8.5, 1.5 Hz), 6.18 (1H, d, J=7.5 Hz), 5.51 (1H, br s), 5.32 (1H, d, J=8.5 Hz), 5.07-5.15 (1H, m), 4.45 (1H, t, J=7.8 Hz), 4.30-4.40 (1H, m), 4.11-4.19 (1H, m), 3.93-4.08 (2H, m), 3.91 (3H, s), 3.27-3.34 (2H, m), 3.26 (3H, s), 2.97-3.08 (2H, m), 2.62 (2H, t, J=5.3 Hz), 2.25-2.42 (2H, m), 1.89-2.14 (4H, m), 1.19-1.83 (17H, m), 1.14 (2H, d, J=6.0 Hz), 0.84-0.90 (6H, m), 0.77-0.84 (3H, m).

### Example 9

### Cyclopentyl (2S)-{[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethyl)carbamoyl]amino}(phenyl)acetate

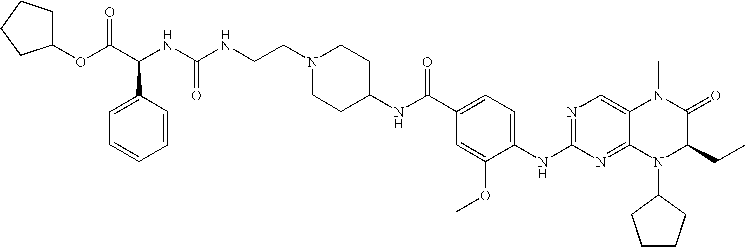

The title compound was prepared from Intermediate C and Intermediate T using the same methodology described for Example 8. LCMS purity 95%; ESMS m/z: 398 [(M+2H)/2]⁺; ¹H NMR (300 MHz, CDCl₃) δ: 8.51 (1H, d, J=8.3 Hz), 7.59-7.66 (2H, m), 7.54 (1H, s), 7.46 (1H, dd, J=5.7, 3.3 Hz), 7.30-7.37 (3H, m), 7.22-7.29 (2H, m), 5.98 (1H, d, J=6.2 Hz), 5.34 (1H, d, J=7.3 Hz), 5.09-5.16 (1H, m), 4.40-4.51 (1H, m), 4.11-4.19 (2H, m), 3.99 (1H, dd, J=6.6, 3.4 Hz), 3.91 (3H, s), 3.29-3.37 (1H, m), 3.26 (3H, s), 2.95-3.11 (1H, m), 2.63 (1H, t, J=6.2 Hz), 2.24-2.42 (2H, m), 1.56-1.82 (10H, m), 1.17-1.47 (10H, m), 0.77-0.88 (9H, m).

### Example 10

### Cyclopentyl (2S)-cyclohexyl{[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethyl)carbamoyl]amino}acetate

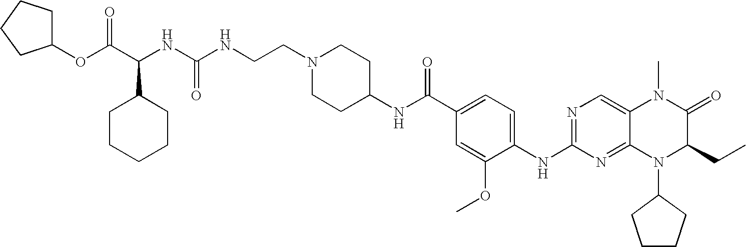

The title compound was prepared from Intermediate C and Intermediate U using the same methodology described for Example 8. LCMS purity 100%; ESMS m/z: 401 [(M+2H)/2]⁺; ¹H NMR (300 MHz, CDCl₃) δ: 7.61 (1H, s), 7.54 (1H, s), 7.46 (1H, s), 7.32 (1H, dd, J=8.7, 1.5 Hz), 6.47 (1H, d, J=8.1 Hz), 5.53 (2H, d, J=8.9 Hz), 5.07-5.16 (1H, m), 4.45 (1H, t, J=7.9 Hz), 4.33 (1H, dd, J=8.9, 4.6 Hz), 4.12-4.19 (2H, m), 3.93-4.01 (1H, m), 3.91 (3H, s), 3.28-3.36 (1H, m), 3.26 (3H, s), 2.95 (2H, t, J=11.8 Hz), 2.53-2.62 (2H, m), 2.15-2.38 (3H, m), 1.93-2.11 (4H, m), 1.55-1.81 (20H, m), 1.18-1.31 (5H, m), 0.76-0.89 (6H, m).

### Example 11

### Cyclopentyl N-[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethoxy)carbonyl]-L-leucinate

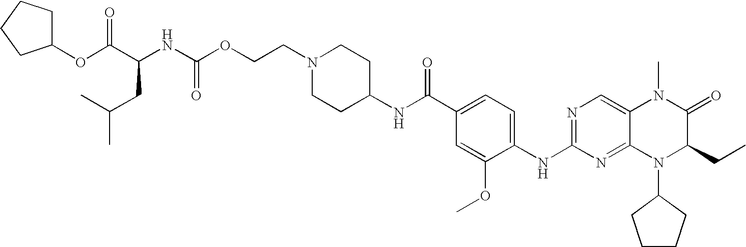

The title compound was prepared by the following methodology (Scheme 9):

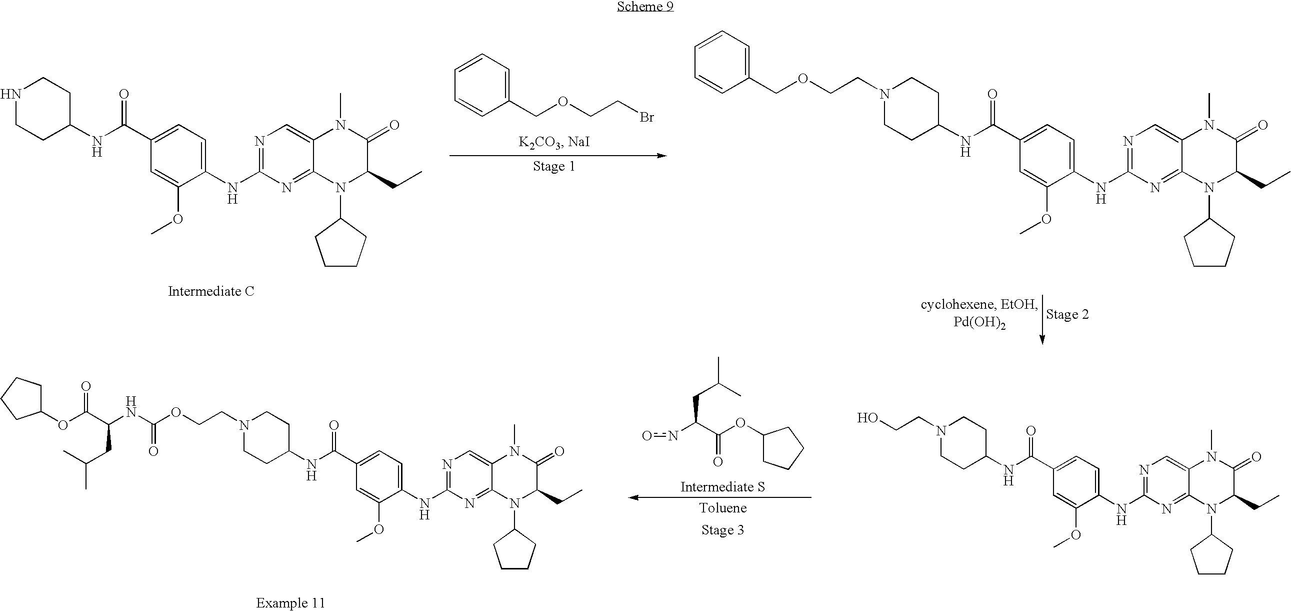

### Stage 1—N-{1-[2-(Benzyloxy)ethyl]piperidin-4-yl}-4-{[(7R)-8-cyclopentyl-7-ethyl-5-methyl-6-oxo-5,6,7,8-tetrahydropteridin-2-yl]amino}-3-methoxybenzamide

To a solution of Intermediate C (300 mg, 0.59 mmol) in DMF (3 ml), was added benzyl-2-bromoethylether (0.09 ml, 0.59 mmol), K₂CO₃ (163 mg, 1.18 mmol) and NaI (catalytic amount). The reaction mixture was stirred for 18 hours at 50° C., cooled to RT and partitioned between EtOAc (10 ml) and water (10 ml). The aqueous layer was re-extracted with EtOAc (2×10 ml) and the combined organic layers were washed with water (2×10 ml), brine (2×15 mL), dried (MgSO₄), and concentrated under reduced pressure. Purification by column chromatography (5-10% MeOH in DCM) afforded the title product (213 mg, 56%). ESMS m/z 642 [M+H]⁺

### Stage 2—4-{[(7R)-8-Cyclopentyl-7-ethyl-5-methyl-6-oxo-5,6,7,8-tetrahydropteridin-2-yl]amino}-N-[1-(2-hydroxyethyl)piperidin-4-yl]-3-methoxybenzamide

To a solution of stage 1 product (134 mg, 0.21 mmol) in cyclohexene (0.13 ml) and EtOH (1.3 ml) was added palladium hydroxide (13 mg). The reaction mixture was stirred at reflux for 4 days during which more cyclohexene and palladium hydroxide were added to drive the reaction to completion. The reaction mixture was cooled down, filtered through Celite® and the filtrate concentrated under reduced pressure to afford the title product (84 mg, 73%). ESMS m/z 552 [M+H]⁺.

### Stage 3—Cyclopentyl N-[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethoxy)carbonyl]-L-leucinate (Example 11)

Stage 2 product (167 mg, 0.30 mmol) was dissolved in toluene (2 ml) and Intermediate S (68 mg, 0.30 mmol) was added. The reaction mixture was stirred at 100° C. for 5 h, cooled down to RT and the solvent removed under reduced pressure. Purification by column chromatography (2 to 5% MeOH in DCM) followed by purification on preparative HPLC afforded the title intermediate (2.5 mg, 11%). LCMS purity 90%; ESMS m/z: 389 [(M+2H)/2]⁺; ¹H NMR (300 MHz, CD₃OD) δ: 8.51 (1H, d, J=9.0 Hz), 7.81 (1H, s), 7.48-7.54 (2H, m), 5.18 (1H, t, J=5.7 Hz), 4.50-4.58 (1H, m), 4.31 (1H, dd, J=7.6, 3.7 Hz), 4.24 (1H, t, J=5.5 Hz), 4.07-4.18 (3H, m), 4.03 (3H, s), 3.34 (3H, s), 3.03-3.14 (2H, m), 2.81-2.87 (1H, m), 2.66-2.79 (3H, m), 2.51 (1H, d, J=16.6 Hz), 2.25-2.38 (2H, m), 1.62-1.80 (16H, m), 1.52-1.61 (3H, m), 1.26 (2H, t, J=7.2 Hz), 0.96 (6H, dd, J=9.5, 6.5 Hz), 0.89 (3H, t, J=7.5 Hz).

### Example 12

### Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}butanoyl)-L-leucinate

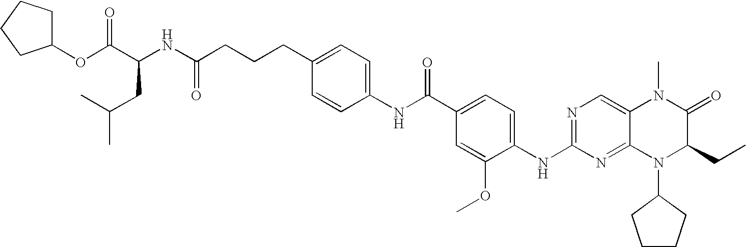

The title compound was prepared by the following methodology (Scheme 10):

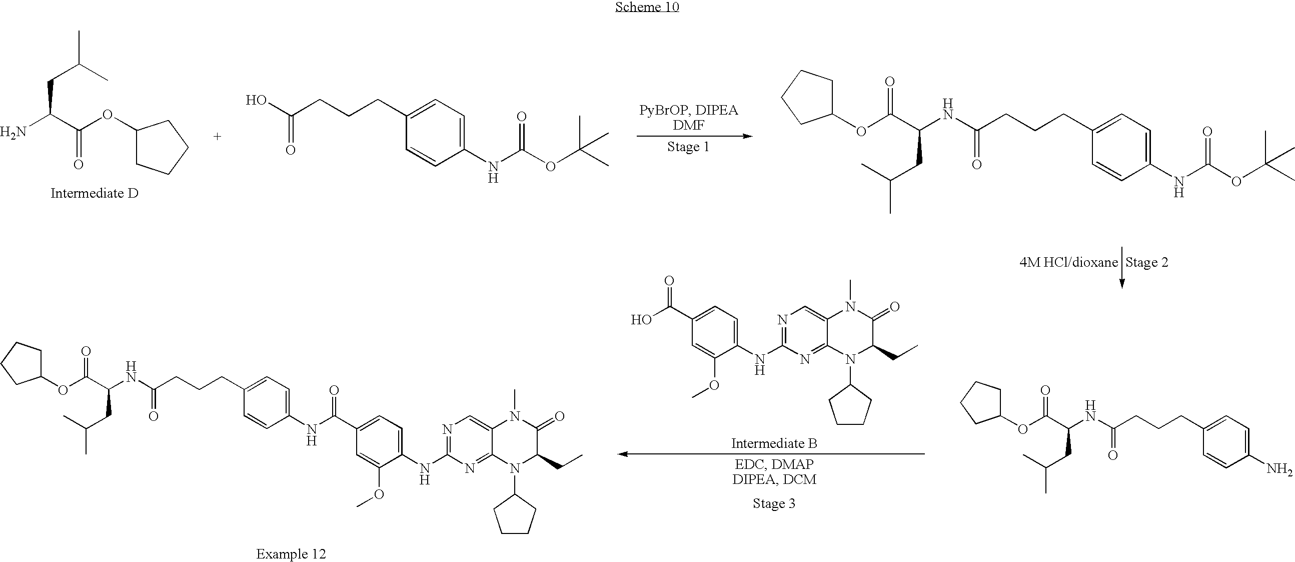

### Stage 1—Cyclopentyl N-(4-{4-[(tert-butoxycarbonyl)amino]phenyl}butanoyl)-L-leucinate

To a solution of Intermediate D (393 mg, 1.97 mmol), 4-{4-[(tert-butoxycarbonyl)amino]phenyl}butanoic acid (500 mg, 1.79 mmol) in DMF (10 ml) was added DIPEA (1 ml, 5.40 mmol) and PyBrOP (1.2 g, 2.69 mmol). The mixture was stirred at RT for 18 hours before dilution with EtOAc (50 ml). The mixture was washed with water (2×50 ml), dried (MgSO₄) and concentrated under reduced pressure. The residue was purified by column chromatography (15% EtOAc in heptane) to afford the title product (410 mg, 50%). ESMS m/z 461 [M+H]⁺.

### Stage 2—Cyclopentyl N-[4-(4-aminophenyl)butanoyl]-L-leucinate

Stage 1 product (410 mg, 0.89 mmol) was dissolved in 4M HCl in dioxane (2 ml) and stirred at RT for 1 hour. The reaction mixture was concentrated under reduced pressure and progressed to the next stage without further purification.

### Stage 3—Cyclopentyl N-(4-{4-[(4-{[(7R)-8-cyclopentyl-7-ethyl-5-methyl-6-oxo-5,6,7,8-tetra hydropteridin-2-yl]amino}-3-methoxybenzoyl)amino]phenyl}butanoyl)-L-leucinate (Example 12)

Stage 2 product (100 mg, 0.27 mmol), Intermediate B (95 mg, 0.22 mmol), EDC (46 mg, 0.24 mmol), DMAP (3 mg, 0.02 mmol) and DIPEA (46 μl, 0.27 mmol) were added to DCM (5 ml) and stirred at RT for 18 hours. The reaction mixture was concentrated under reduced pressure and purified by column chromatography (50-100% EtOAc in heptane) to afford the title intermediate (40 mg, 19%). LCMS purity 95%; ESMS m/z: 768 [M+H]⁺; ¹H NMR (300 MHz, CDCl₃) δ: 8.33 (1H, br s), 8.25 (1H, br s), 7.60 (2H, d, J=8.3 Hz), 7.50 (1H, s), 7.36 (2H, dd, J=1.4, 8.5 Hz), 7.10 (2H, d, J=8.2 Hz), 5.85 (1H, d, J=8.4 Hz), 5.14-5.11 (1H, m), 4.53-4.48 (1H, m), 4.39 (1H, t, J=7.9 Hz), 4.23-4.19 (1H, m), 3.95 (3H, s), 3.15 (3H, s), 2.56 (2H, t, J=7.4 Hz), 2.24-1.67 (25H, m), 0.88 (6H, d, J=5.8 Hz) 0.80 (3H, t, J=7.4 Hz).

### Example 13

### Cyclopentyl N-{4-[(4-{[(7R)-8-cyclopentyl-7-ethyl-5-methyl-6-oxo-5,6,7,8-tetrahydropteridin-2-yl]amino}-3-methoxybenzoyl)amino]butanoyl}-L-leucinate

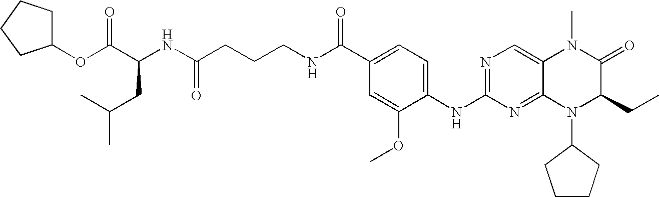

The title compound was prepared by the following methodology (Scheme 11):

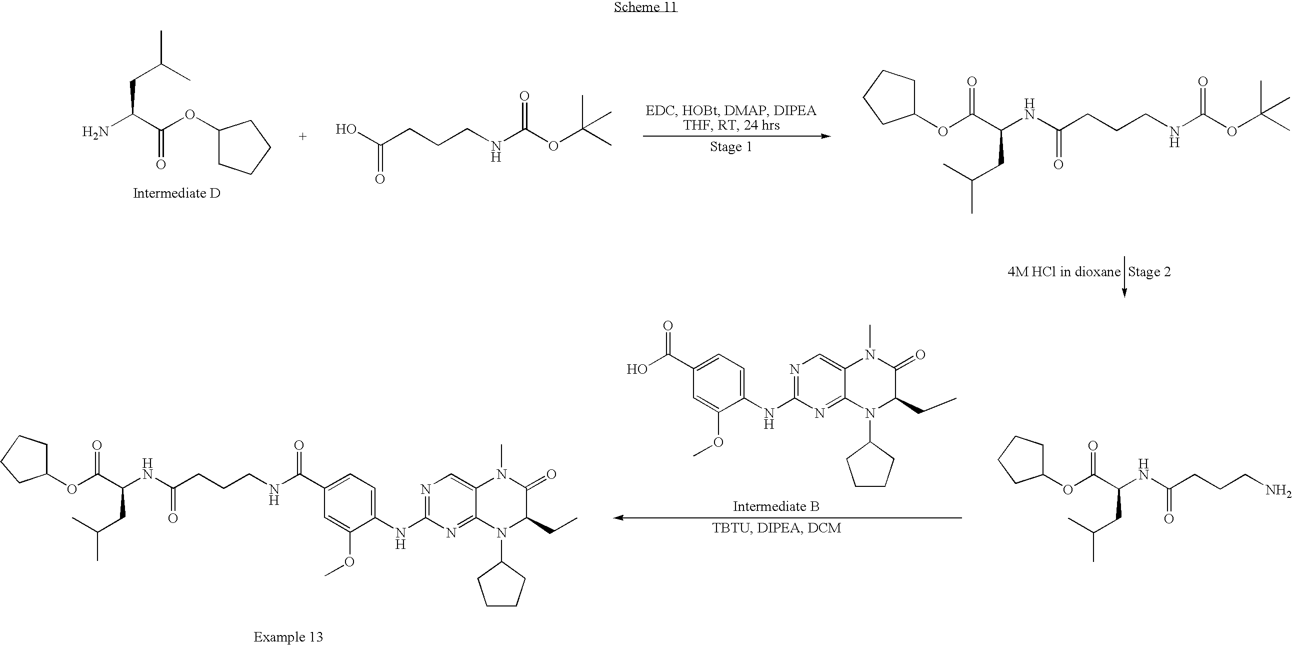

### Stage 1—Cyclopentyl N-{4-[(tert-butoxycarbonyl)amino]butanoyl}-L-leucinate

To a stirred solution of Intermediate D (212 mg, 1.06 mmol) in THF (5 ml) was added 4-(t-butoxycarbonylamino)butyric acid (144 mg, 0.71 mmol), HOBt (105 mg, 0.78 mmol), DMAP (8.6 mg, 0.07 mmol) and DIPEA (0.31 ml, 1.77 mmol). The solution was cooled to 0° C. and EDC (150 mg, 0.78 mmol) was added. The reaction mixture was warmed to RT and stirred for a further 18 hours. The reaction mixture was diluted with EtOAc (10 ml) and washed with water (2×10 ml). The organic layers were dried (MgSO₄) and concentrated under reduced pressure. The crude product was purified by column chromatography (20% EtOAc in heptane) to afford the title product as a colourless oil (253 mg, 93%).

### Stage 2—Cyclopentyl N-(4-aminobutanoyl)-L-leucinate

Stage 1 product (253 mg, 0.65 mmol) was dissolved in 4M HCl in dioxane (3 ml) and stirred at RT for 1 h. The reaction mixture was concentrated under reduced pressure and progressed to the next stage without further purification.

### Stage 3—Cyclopentyl N-{4-[(4-{[(7R)-8-cyclopentyl-7-ethyl-5-methyl-6-oxo-5,6,7,8-tetrahydro-pteridin-2-yl]amino}-3-methoxybenzoyl)amino]butanoyl}-L-leucinate (Example 13)

To a solution of Intermediate B (132 mg, 0.31 mmol) in DCM (2 ml) was added DIPEA (0.22 ml, 1.24 mmol) and TBTU (110 mg, 0.34 mmol) and the reaction stirred at RT for 30 min. Stage 2 product (106 mg, 0.37 mmol) was added to the reaction mixture and the mixture stirred at RT for 50 min before dilution with DCM (5 ml). The mixture was washed with water (2×10 ml) and brine (2×10 ml). The organic layer was dried (MgSO₄), and concentrated under reduced pressure. The crude product was purified by column chromatography (20%-50 EtOAc in heptane) to afford the title product as a colourless oil which solidified on scratching. (87.2 mg, 41%). LCMS purity 96%; ESMS m/z: 692 [M+H]⁺; ¹H NMR (300 MHz, CDCl₃) δ: 8.45 (1H, d, J=8.5 Hz), 7.57-7.61 (2H, m), 7.27-7.35 (1H, m), 7.06 (1H, t, J=5.2 Hz), 6.48 (1H, d, J=8.1 Hz), 5.05-5.14 (1H, m), 4.36-4.54 (2H, m), 4.11-4.19 (1H, m), 3.90 (3H, s), 3.67 (2H, t, J=6.5 Hz), 3.36-3.54 (2H, m), 3.25 (3H, s), 2.25-2.35 (2H, m), 1.56-1.96 (20H, m), 0.70-0.94 (11H, m).

### Example 14

### Cyclopentyl N-[(5E)-6-(4-{[(7R)-8-cyclopentyl-7-ethyl-5-methyl-6-oxo-5,6,7,8-tetrahydropteridin-2-yl]amino}-3-fluorophenyl)hex-5-enoyl]-L-leucinate

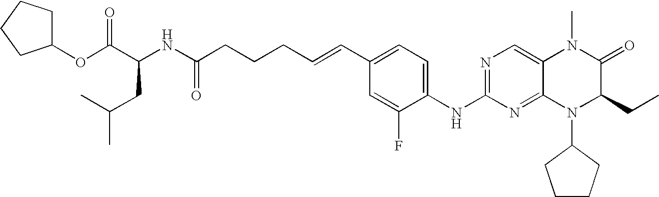

The title compound was prepared by the following methodology (Scheme 12):

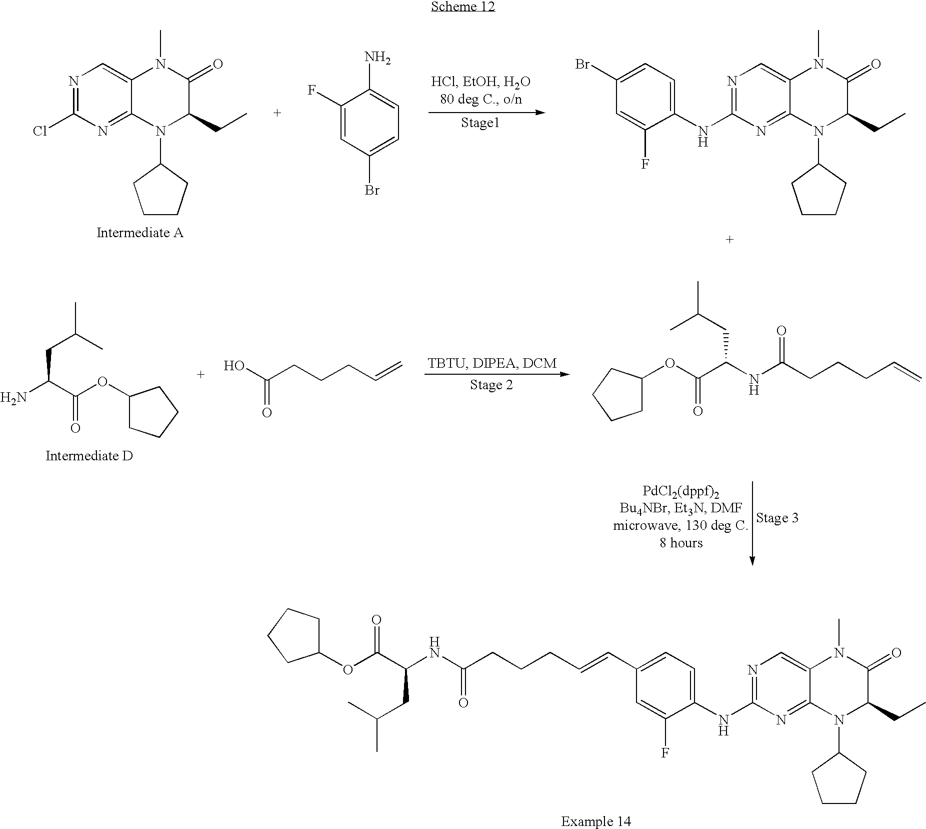

### Stage 1-(7R)-2-[(4-Bromo-2-fluorophenyl)amino]-8-cyclopentyl-7-ethyl-5-methyl-7,8-dihydropteridin-6(5H)-one

To a suspension of 4-bromo-2-fluoro-phenylamine (388 mg, 2.04 mmol) in ethanol (2.5 ml) and water (10 ml) was added concentrated HCl (0.26 ml) and intermediate A (300 mg, 1.02 mmol). The mixture was heated at 80° C. for 18 hours, cooled and the solvent was removed under reduced pressure. The residue was diluted with EtOAc (10 ml) and washed with saturated NaHCO₃ (2×10 ml). The organic layer was dried (MgSO₄) and concentrated under reduced pressure. The crude product was purified by column chromatography (20-50% EtOAc in heptane) to afford the title product as a yellow oil (90 mg, 20%). ESMS: m/z 448, 449, 450 [Br splitting].

### Stage 2—Cyclopentyl N-hex-5-enoyl-L-leucinate

Procedure as in Stage 1 Intermediate R

### Stage 3—Cyclopentyl N-[(5E)-6-(4-{[(7R)-8-cyclopentyl-7-ethyl-5-methyl-6-oxo-5,6,7,8-Tetrahydropteridin-2-yl]amino}-3-fluorophenyl)hex-5-enoyl]-L-leucinate (Example 14)

To a solution of Stage 1 product (90 mg, 0.20 mmol) in DMF (1 ml) was added Stage 2 product (90 mg, 0.30 mmol), PdCl₂(dppf)₂ (16.3 mg, 0.02 mmol), tetrabutylammonium bromide (65 mg, 0.20 mmol) and Et₃N (0.06 ml, 0.44 mmol). The reaction mixture was heated in the microwave at 130° C. for 8 h. The resulting mixture was diluted with EtOAc (10 ml) and dry loaded onto silica. The crude product was purified up by column chromatography (20-50% EtOAc in heptane) before a final purification by preparative HPLC afforded the title example as a colourless oil. (11.2 mg, 8.4%). LCMS purity 92%; ESMS m/z: 663 [M+H]⁺; ¹H NMR (300 MHz, CD₃OD) δ: 7.07-7.59 (4H, m), 6.38-6.52 (1H, m), 5.13-5.22 (1H, m), 4.43-4.51 (1H, m), 4.38 (1H, t, J=7.4 Hz), 3.99-4.06 (1H, m), 3.30 (3H, s), 1.33-2.42 (28H, m), 0.96 (6H, dd, J=13.0, 6.4 Hz), 0.85 (3H, t, J=7.3 Hz).

### Example 15

### Cyclopentyl N-[6-(4-{[(7R)-8-cyclopentyl-7-ethyl-5-methyl-6-oxo-5,6,7,8-tetrahydropteridin-2-yl]amino}-3-fluorophenyl)hexanoyl]-L-leucinate

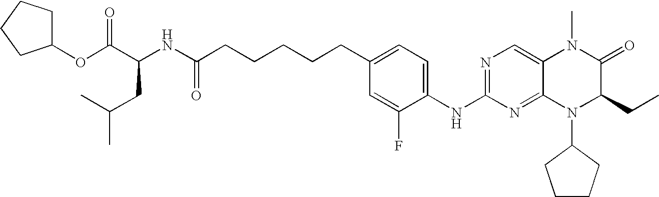

The title compound was prepared by the following methodology (Scheme 13)

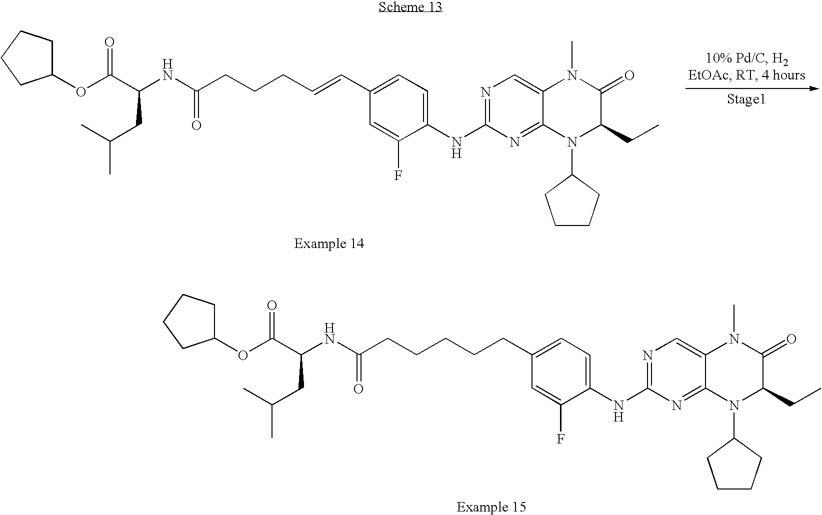

### Stage 1—Cyclopentyl N-[6-(4-{[(7R)-8-cyclopentyl-7-ethyl-5-methyl-6-oxo-5,6,7,8-tetrahydropteridin-2-yl]amino}-3-fluorophenyl)hexanoyl]-L-leucinate (Example 15)

This stage is the same as in Stage 1 Intermediate R

LCMS purity 99%; ESMS m/z: 665 [M+H]⁺; ¹H NMR (300 MHz, CDCl₃) δ: 11.0 (1H, s), 7.3 (2H, d, J=6.8 Hz), 6.95 (2H, d, J=9.4 Hz), 5.81-5.94 (1H, m), 5.1-5.3 (1H, m), 4.6 (1H, td, J=8.4, 5.3 Hz), 4.3 (1H, dd, J=6.4, 3.0 Hz), 3.7-3.9 (1H, m), 2.6 (2H, t, J=7.6 Hz), 2.24 (2H, t, J=7.5 Hz), 1.55-1.9 (20H, m), 1.3-1.4 (6H, m), 0.96 (6H, d, J=6.0 Hz), 0.85 (3H, t, J=7.4 Hz).

### Example 16

### Cyclopentyl N-[(5E)-6-(4-{[(7R)-8-cyclopentyl-7-ethyl-5-methyl-6-oxo-5,6,7,8-tetrahydropteridin-2-yl]amino}-3,5-difluorophenyl)hex-5-enoyl]-L-leucinate

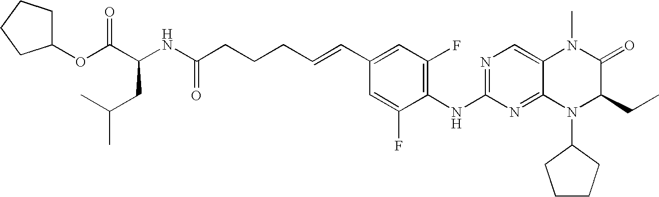

The title compound was prepared by the following methodology (Scheme 14):

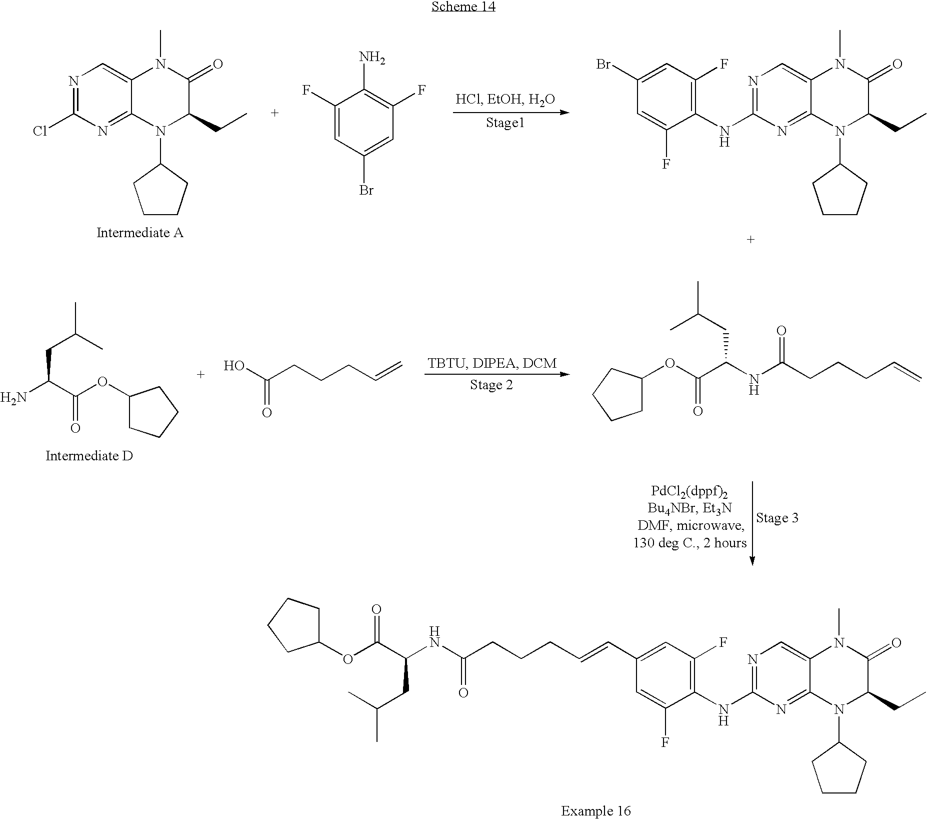

### Stage 1—(7R)-2-[(4-Bromo-2,6-difluorophenyl)amino]-8-cyclopentyl-7-ethyl-5-methyl-7,8-dihydropteridin-6(5H)-one

Procedure as in Stage 1 Example 14 using 4-Bromo-2,6-difluoro-phenylamine

ESMS m/z: 466, 467, 468 [Br splitting].

### Stage 2—Cyclopentyl N-hex-5-enoyl-L-leucinate

Procedure as in Stage 2 in Example 14.

### Stage 3—Procedure as in Stage 3 in Example 14

LCMS purity 91%; ESMS m/z: 681 [M+H]⁺; ¹H NMR (300 MHz, CDCl₃) δ: 7.22-7.29 (1H, m), 6.86 (2H, d, J=8.9 Hz), 6.08-6.29 (1H, m), 5.75-5.85 (1H, m), 5.08-5.18 (1H, m), 4.45-4.58 (1H, m), 4.19-4.27 (1H, m), 3.64-3.80 (1H, m), 3.20 (3H, s), 1.24-2.23 (27H, m), 0.88 (6H, d, J=5.3 Hz), 0.77 (3H, t, J=7.3 Hz).

### Example 17

### Cyclopentyl N-(3-{cis-4-[(4-{[(7R)-8-cyclopentyl-7-ethyl-5-methyl-6-oxo-5,6,7,8-tetrahydropteridin-2-yl]amino}-3-methoxybenzoyl)amino]cyclohexyl}propanoyl)-L-leucinate

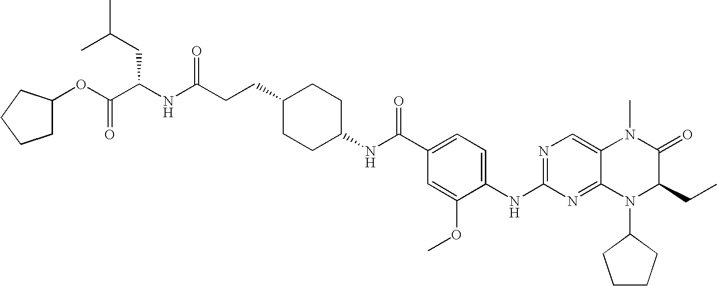

The title compound was prepared by the following methodology (Scheme 15):

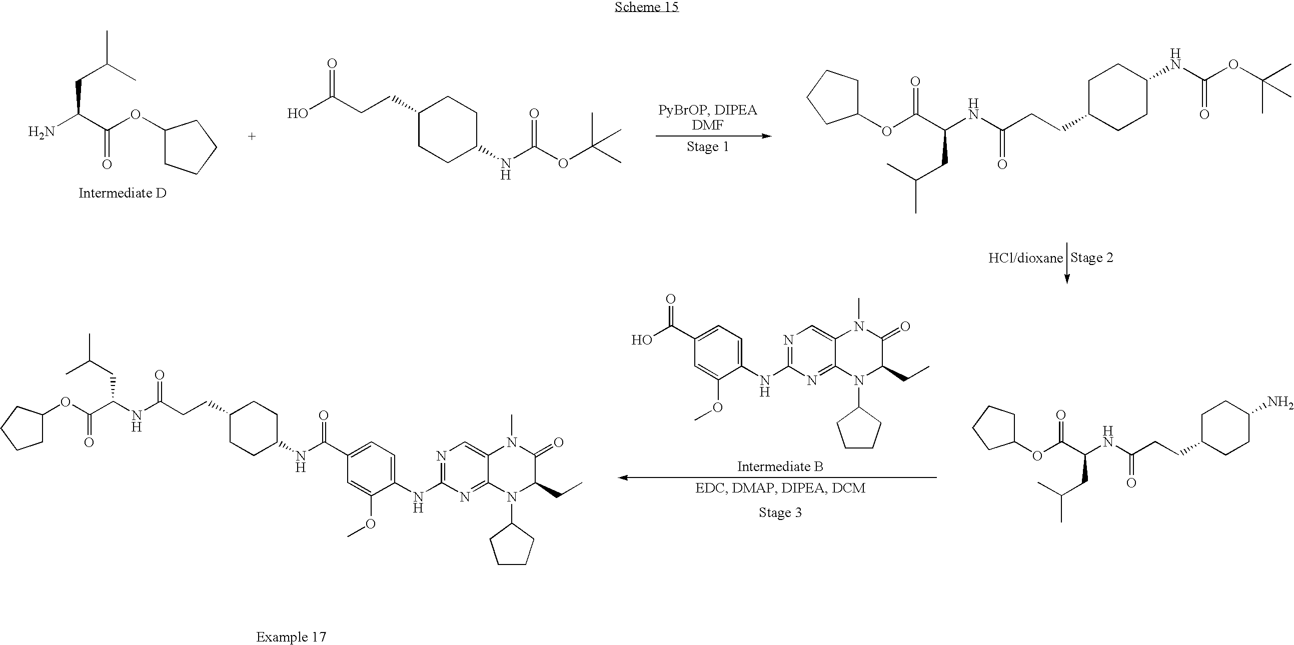

### Stage 1—Cyclopentyl N-(3-{cis-4-[(tert-butoxycarbonyl)amino]cyclohexyl}propanoyl)-L-leucinate

To a solution of Intermediate D (24 mg, 0.12 mmol) in DMF(2 ml) was added 3-{cis-4-[(tert-butoxycarbonyl)amino]cyclohexyl}propanoic acid (30 mg, 0.11 mmol), PyBrOP (85 mg, 0.18 mmol) and DIPEA (60 μl, 0.37 mmol). The reaction mixture was stirred at RT for 18 hours before dilution with EtOAc (150 ml). The mixture was washed with water (3×50 ml), dried (MgSO₄) and concentrated under reduced pressure. The residue was used directly in the next stage without further purification.

### Stage 2—Cyclopentyl N-[3-(cis-4-aminocyclohexyl)propanoyl]-L-leucinate

Crude Stage 1 product was dissolved in 4M HCl in dioxane (1 ml) and stirred at RT for 1 hour. The reaction mixture was then concentrated in vacuo and the residue progressed to the next step without further purification.

### Stage 3—Cyclopentyl N-(3-{cis-4-[(4-{[(7R)-8-cyclopentyl-7-ethyl-5-methyl-6-oxo-5,6,7,8-tetrahydropteridin-2-yl]amino}-3-methoxybenzoyl)amino]cyclohexyl}propanoyl)-L-leucinate (Example 17)

To a solution of Stage 2 product (39 mg, 0.11 mmol) in DCM (2 ml) was added Intermediate B (47 mg, 0.11), EDC (23 mg, 0.12 mmol), DMAP (1 mg, 0.01 mmol) and DIPEA (23 μl, 0.13 mmol). The reaction mixture was stirred at RT for 18 hours before being concentrated under reduced pressure. Purification by HPLC to afford the title example (30 mg, 36%). LCMS purity 95%; ESMS m/z: 760 [M+H]⁺; ¹H NMR (300 MHz, CD₃OD) δ: 8.50 (1H, d, J=9.0 Hz), 7.80 (1H, s), 7.49-4.48 (2H, m), 5.18 (1H, m), 4.60-4.52 (1H, m), 4.38 (1H, dd, J=6.6, 8.9 Hz), 4.30 (1H, dd, J=3.8, 7.7 Hz), 4.02 (3H, s), 2.29 (2H, t, J=6.9 Hz), 2.02-1.57 (25H, m), 1.00-0.86 (10H, t, J=7.4 Hz).

### Example 18

### N-(4-{4-[(4-{[(7R)-8-Cyclopentyl-7-ethyl-5-methyl-6-oxo-5,6,7,8-tetrahydropteridin-2-yl]amino}-3-methoxybenzoyl)amino]piperidin-1-yl}butanoyl)-L-leucine

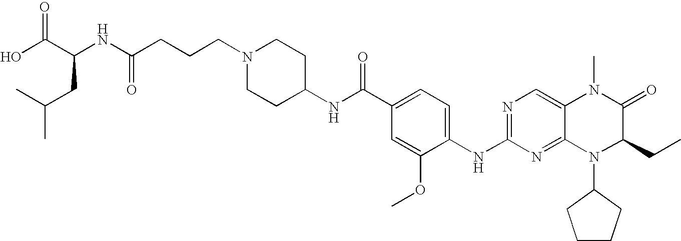

The title compound was prepared by the following methodology (Scheme 16):

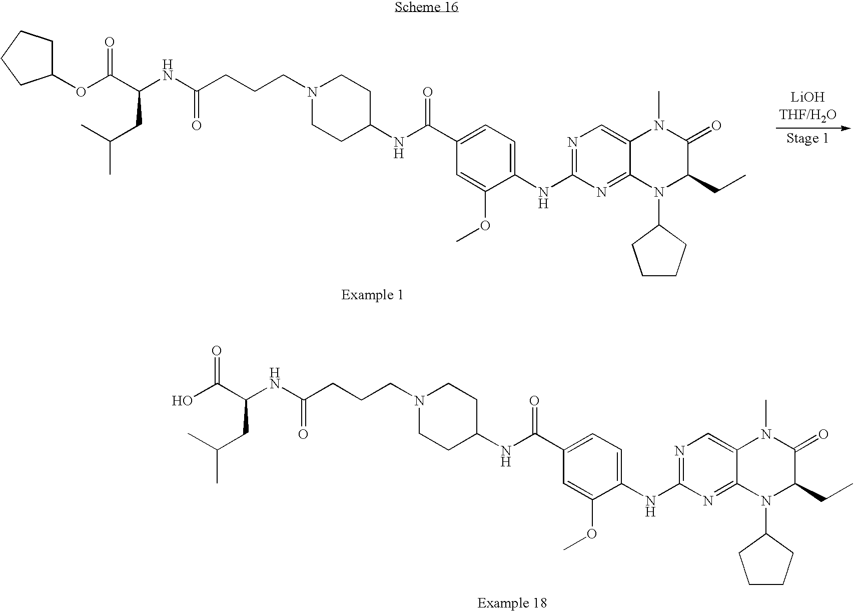

### Stage 1—N-(4-{4-[(4-{[(7R)-8-Cyclopentyl-7-ethyl-5-methyl-6-oxo-5,6,7,8-tetrahydropteridin-2-yl]amino}-3-methoxybenzoyl)amino]piperidin-1-yl}butanoyl)-L-leucine (Example 18)

To a solution of Example 1 (35 mg, 0.05 mmol) in THF (1 ml) and water (1 ml) was added LiOH (25 mg, 1.03 mmol). The reaction mixture was stirred at RT for 4 hours and concentrated under reduced pressure. The residue was taken up in water (10 ml) and acidified to pH ˜6 with 1 M HCl and extracted with n-butanol (3×10 ml). The combined organic extracts were concentrated under reduced pressure and the residue was triturated with Et₂O to afford the title product as an off-white solid (30 mg, 95%). LCMS purity 93%; ESMS m/z: 609 [M+H]⁺; ¹H NMR (300 MHz, d₆-DMSO) δ: 9.42 (1H, br s), 8.44-8.39 (2H, m), 7.85 (1H, s), 7.61 (1H, s), 7.55-7.53 (2H, m), 4.39-4.33 (2H, m), 4.24 (1H, d, J=3.5, 7.4 Hz), 4.14-4.00 (1H, m), 3.96 (3H, s), 3.66 (1H, d, J=7.5 Hz), 3.25 (3H, s), 3.24-3.14 (2H, m), 3.13-2.96 (1H, m), 2.91-2.80 (1H, m), 2.21-1.61 (19H, m), 0.76 (3H, t, J=7.4 Hz).

### Example 19

### N-(4-{4-[(4-{[(7R)-8-Cyclopentyl-7-ethyl-5-methyl-6-oxo-5,6,7,8-tetrahydropteridin-2-yl]amino}-3-methoxybenzoyl)amino]Piperidin-1-yl}butanoyl)-D-leucine

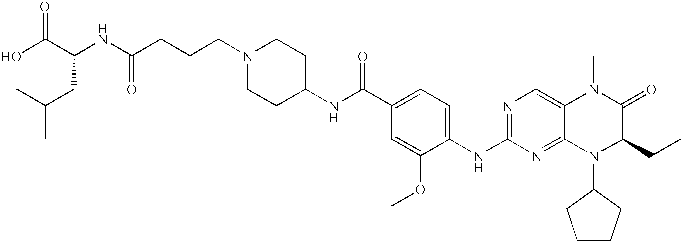

The title compound was prepared from Example 2 using the same methodology described for Example 18. LCMS purity 85%; ESMS m/z: 775 [M+H]⁺; ¹H NMR (300 MHz, CD₃OD) δ: 8.01 (1H, d, J=7.7 Hz), 7.64-7.60 (3H, m), 4.47-4.34 (3H, m), 4.29 (1H, m), 4.02 (3H, s), 3.34 (3H, s), 3.25 (2H, m), 2.52 (2H, m), 2.24-1.67 (23H, m), 1.00 (3H, d, J=6.0 Hz), 0.96 (3H, d, J=6.2 Hz) 0.88 (3H, t, J=7.5 Hz).

### Example 20

### (2S)-[(4-{4-[(4-{[(7R)-8-Cyclopentyl-7-ethyl-5-methyl-6-oxo-5,6,7,8-tetrahydropteridin-2-yl]amino}-3-methoxybenzoyl)amino]piperidin-1-yl}butanoyl)amino](phenyl)acetic acid

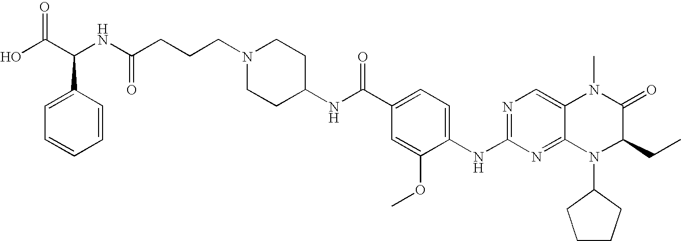

The title compound was prepared from Example 3 using the same methodology described for Example 18. LCMS purity 95%; ESMS m/z: 726 [M+H]⁺; ¹H NMR (300 MHz, CD₃OD) δ: 8.29 (1H, d, J=8.0 Hz), 7.76 (1H, m), 7.59 (2H, m), 7.46-7.36 (5H, m), 5.42 (1H, s), 4.49-4.44 (1H, m), 4.39-4.38 (1H, m), 4.03 (3H, s), 3.93-3.89 (1H, m) 3.31 (3H, s), 3.26 (2H, m), 2.56 (2H, m), 2.23-1.31 (20H, m), 0.88 (3H, t, J=7.4 Hz).

### Example 21

### (2R)-[(4-{4-[(4-{[(7R)-8-Cyclopentyl-7-ethyl-5-methyl-6-oxo-5,6,7,8-tetrahydropteridin-2-yl]amino}-3-methoxybenzoyl)amino]piperidin-1-yl}butanoyl)amino](phenyl)acetic acid

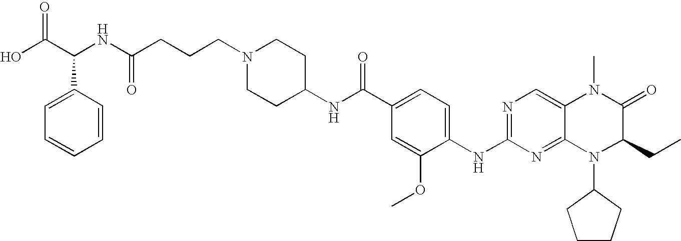

The title compound was prepared from Example 4 using the same methodology described for Example 18. LCMS purity 85%; ESMS m/z: 727 [M+H]⁺; ¹H NMR (300 MHz, CD₃OD) δ: 8.03 (1H, d, J=8.4 Hz), 7.70-7.57 (3H, m), 7.43-7.38 (5H, m), 5.46 (1H, s), 4.46 (1H, m), 4.36 (1H, m), 4.22 (1H, m), 4.02 (3H, s) 3.37 (3H, s), 3.10 (2H, m), 2.55-2.53 (2H, m), 2.24-1.66 (20H, m), 0.88 (3H, t, J=7.4 Hz).

### Example 22

### (2R)-Cyclohexyl[(4-{4-[(4-{[(7R)-8-cyclopentyl-7-ethyl-5-methyl-6-oxo-5,6,78-tetrahydropteridin-2-yl]amino}-3-methoxybenzoyl)amino]piperidin-1-yl}butanoyl)amino]acetic acid

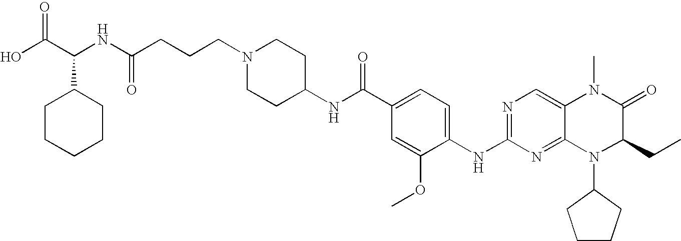

The title compound was prepared from Example 5 using the same methodology described for Example 18. LCMS purity 95%; ESMS m/z: 733 [M+H]⁺; ¹H NMR (300 MHz, CD₃OD) δ: 8.11 (1H, d, J=8.5 Hz), 7.69-7.55 (2H, m), 7.36 (1H, m), 4.43-4.22 (3H, m), 4.01 (3H, s), 3.73-3.69 (1H, m), 3.34 (3H, s), 2.59 (2H, m), 2.27-1.70 (28H, m), 1.31-1.15 (5H, m), 0.88 (3H, t, J=7.5 Hz).

### Example 23

### N-(5-[4-[(4-{[(7R)-8-Cyclopentyl-7-ethyl-5-methyl-6-oxo-5,6,7,8-tetrahydropteridin-2-yl]amino}-3-methoxybenzoyl)amino]piperidin-1-yl]pentanoyl)-L-leucine

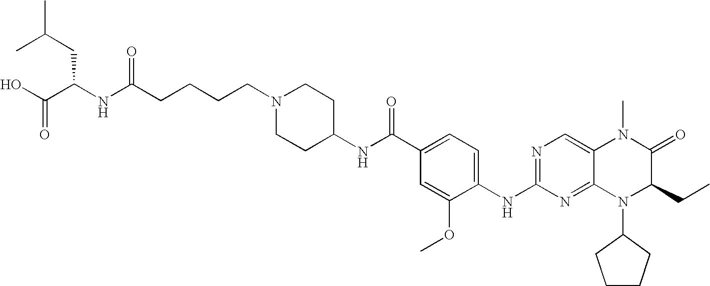

The title compound was prepared from Example 6 using the same methodology described for Example 18. LCMS purity 100%; ESMS m/z: 721 [M+H]⁺; ¹H NMR (300 MHz, d₆-DMSO) δ: 8.41 (1H, d, J=8.9 Hz), 8.05-8.13 (1H, m), 7.84 (1H, s), 7.59 (1H, s), 7.44-7.52 (1H, m), 4.28-4.40 (2H, m), 4.23 (1H, dd, J=7.3, 3.8 Hz), 3.94 (3H, s), 3.24 (3H, s), 2.83-2.93 (2H, m), 2.24-2.32 (2H, m), 2.09-2.17 (2H, m), 1.70-1.92 (9H, m), 1.57-1.67 (5H, m), 1.02-1.52 (10H, m), 0.81-0.92 (8H, m), 0.76 (3H, t, J=7.4 Hz).

### Example 24

### N-({4-[(4-{[(7R)-8-Cyclopentyl-7-ethyl-5-methyl-6-oxo-5,6,7,8-tetrahydropteridin-2-yl]amino}-3-methoxybenzoyl)amino]piperidin-1-yl}acetyl)-L-leucine

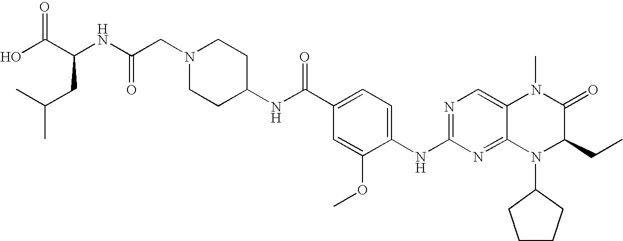

The title compound was prepared from Example 7 using the same methodology described for Example 18. LCMS purity 93%; ESMS m/z: 679 [M+H]⁺; ¹H NMR (300 MHz, CD₃OD) δ: 8.07 (1H, d, J=8.3 Hz), 7.68 (1H, s), 7.61 (1H, d, J=1.5 Hz), 7.57 (1H, dd, J=8.3, 1.5 Hz), 4.31-4.51 (4H, m), 4.21 (1H, t, J=10.5 Hz), 4.02 (1H, br s), 4.00 (3H, s), 3.61-3.74 (2H, m), 3.35 (3H, s), 2.18-2.29 (2H, m), 1.80-2.16 (11H, m), 1.55-1.79 (6H, m), 0.93-1.01 (6H, m), 0.86 (3H, t, J=7.5 Hz).

### Example 25

### (2S)-Cyclohexyl{[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethyl)carbamoyl]amino}acetic acid

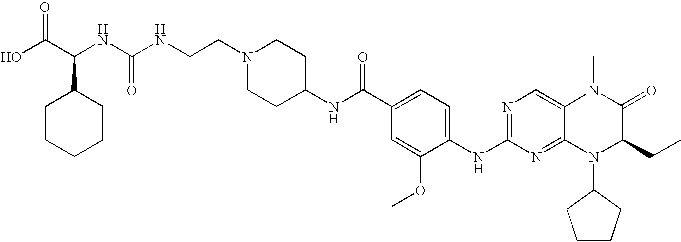

The title compound was prepared from Example 10 using the same methodology described for Example 18. LCMS purity 90%; ESMS m/z: 367 [(M+2H)/2]⁺; ¹H NMR (300 MHz, CD₃OD) δ: 8.47-8.55 (1H, m), 7.81 (1H, br s), 7.47-7.57 (2H, m), 4.54 (1H, t, J=7.8 Hz), 4.31 (1H, dd, J=7.5, 3.6 Hz), 4.02 (3H, s), 3.99 (1H, d, J=3.4 Hz), 3.74 (1H, t, J=6.2 Hz), 3.35 (3H, s), 2.84-2.93 (2H, m), 2.58 (2H, m), 1.99-2.26 (4H, m), 1.46-1.97 (19H, m), 1.09-1.42 (6H, m), 0.88 (3H, t, J=7.4 Hz).

### Example 26

### N-(4-{4-[(4-{[(7R)-8-Cyclopentyl-7-ethyl-5-methyl-6-oxo-5,6,7,8-tetrahydropteridin-2-yl]amino}-3-methoxybenzoyl)amino]phenyl}butanoyl)-L-leucine

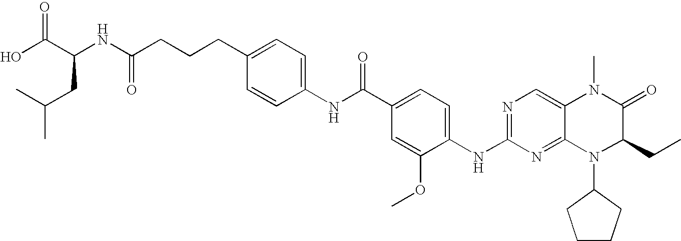

The title compound was prepared from Example 12 using the same methodology described for Example 18. LCMS purity 95%; ESMS m/z: 700 [M+H]⁺; ¹H NMR (300 MHz, CD₃OD) δ: 8.03 (1H, d, J=8.2 Hz), 7.71-7.60 (5H, m), 7.24 (2H, d, J=8.5 Hz), 4.47-4.36 (3H, m), 4.03 (3H, s), 3.33 (3H, s), 2.70-2.64 (2H, m), 2.33-2.28 (2H, m), 1.98-1.63 (15H, m), 1.00-0.95 (6H, m) 0.89 (3H, t, J=7.58 Hz).

### Example 27

### N-{4-[(4-{[(7R)-8-Cyclopentyl-7-ethyl-5-methyl-6-oxo-5,6,7,8-tetrahydropteridin-2-yl]amino}-3-methoxybenzoyl)amino]butanoyl}-L-leucine

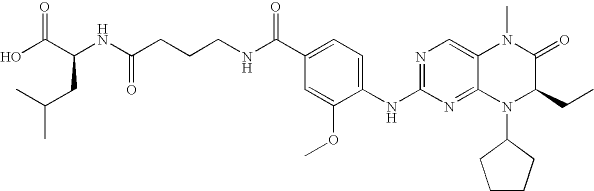

The title compound was prepared from Example 13 using the same methodology described for Example 18. LCMS purity 99%; ESMS m/z: 624 [M+H]⁺; ¹H NMR (300 MHz, CD₃OD) δ: 8.40-8.54 (2H, m), 7.48-7.81 (2H, m), 4.35-4.60 (2H, m), 4.25-4.33 (1H, m), 4.01 (3H, s), 3.38-3.51 (2H, m), 3.32 (3H, s), 2.37 (2H, t, J=6.8 Hz), 2.08-2.24 (1H, m), 1.50-2.06 (14H, m), 0.74-1.04 (9H, m).

### Example 28

### N-[(5E)-6-(4-{[(7R)-8-Cyclopentyl-7-ethyl-5-methyl-6-oxo-5,6,7,8-tetrahydropteridin-2-yl]amino}-3-fluorophenyl)hex-5-enoyl]-L-leucine

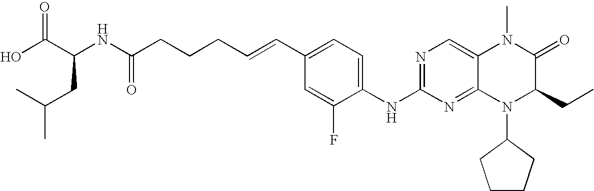

The title compound was prepared from Example 14 using the same methodology described for Example 18. LCMS purity 95%; ESMS m/z: 595 [M+H]⁺; ¹H NMR (300 MHz, CD₃OD) δ: 7.23-7.58 (4H, m), 6.32-6.52 (1H, m), 4.45 (2H, dd, J=6.2, 3.2 Hz), 4.00-4.13 (1H, m), 3.31 (3H, s), 1.31-2.71 (20H, m), 0.97 (6H, dd, J=10.0, 6.2 Hz), 0.85 (3H, t, J=7.4 Hz).

### Example 29

### N-[6-(4-{[(7R)-8-Cyclopentyl-7-ethyl-5-methyl-6-oxo-5,6,7,8-tetrahydropteridin-2-yl]amino}-3-fluorophenyl)hexanoyl]-L-leucine

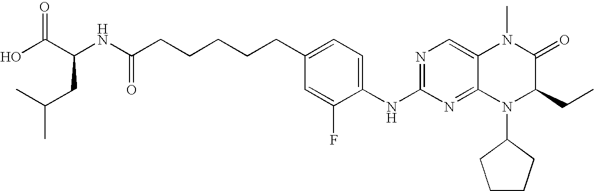

The title compound was prepared from Example 15 using the same methodology described for Example 18. LCMS purity 97%; ESMS m/z: 597 [M+H]⁺; ¹H NMR (300 MHz, CD₃OD) δ: 7.71 (2H, t, J=8.5 Hz), 6.89-7.11 (2H, m), 5.02 (1H, s), 4.42 (1H, br s), 4.11-4.31 (2H, m), 2.56-2.67 (2H, m), 2.26 (2H, t, J=7.3 Hz), 1.48-2.03 (18H, m), 0.95 (6H, dd, J=11.0, 6.1 Hz), 0.85 (3H, t, J=7.4 Hz).

### Example 30

### N-[(5E)-6-(4-{[(7R)-8-Cyclopentyl-7-ethyl-5-methyl-6-oxo-5,6,7,8-tetrahydropteridin-2-yl]amino}-3,5-difluorophenyl)hex-5-enoyl]-L-leucine

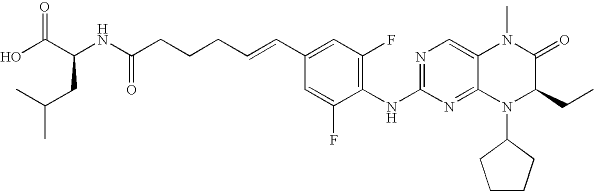

The title compound was prepared from Example 16 using the same methodology described for Example 18. LCMS purity 97%; ESMS m/z: 613 [M+H]⁺; ¹H NMR (300 MHz, CD₃OD) δ: 7.52 (1H, br s), 6.98-7.25 (2H, m), 6.46 (1H, s), 4.39-4.51 (2H, m), 3.84-4.00 (1H, m), 3.31 (3H, s), 1.31-2.38 (20H, m), 0.97 (6H, dd, J=10.4, 6.2 Hz), 0.84 (3H, t, J=7.4 Hz).

### Example 31

### N-(3-{cis-4-[(4-{[(7R)-8-Cyclopentyl-7-ethyl-5-methyl-6-oxo-5,6,7,8-tetrahydropteridin-2-yl]amino}-3-methoxybenzoyl)amino]cyclohexyl}propanoyl)-L-leucine

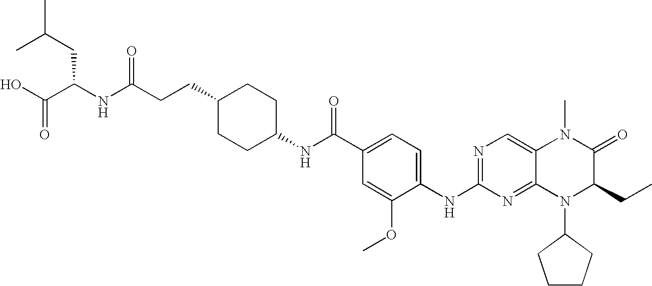

The title compound was prepared from Example 17 using the same methodology described for Example 18. LCMS purity 95%; ESMS m/z: 692 [M+H]⁺; ¹H NMR (300 MHz, CD₃OD) δ: 7.76 (1H, d, J=8.4 Hz), 7.48 (2H, d, J=1.9 Hz), 7.43 (1H, dd, J=1.9, 8.2 Hz), 4.39-4.31 (2H, m), 4.22 (1H, t, J=9.7 Hz), 3.91 (1H, m), 3.88 (3H, s), 3.25 (3H, s), 2.19 (2H, t, J=7.2 Hz), 1.95-1.81 (6H, m), 1.59-1.48 (18H, m), 0.85 (6H, dd, J=6.3, 11.5 Hz), 0.76 (3H, t, J=7.5 Hz).

### Biological Assays

### Broken Cell Carboxylesterase Assay

Any given compound of the present invention wherein R₄ is an ester group, may be tested to determine whether it meets the requirement that it be hydrolysed by intracellular esterases, by testing in the following assay.

### Preparation of Cell Extract

U937 or HCT116 tumour cells (˜10⁹) were washed in 4 volumes of Dulbeccos PBS (˜1 litre) and pelleted at 525 g for 10 min at 4° C. This was repeated twice and the final cell pellet was resuspended in 35 ml of cold homogenising buffer (Trizma 10 mM, NaCl 130 mM, CaCl₂ 0.5 mM pH 7.0 at 25° C.). Homogenates were prepared by nitrogen cavitation (700 psi for 50 min at 4° C.). The homogenate was kept on ice and supplemented with a cocktail of inhibitors at final concentrations of:
Leupeptin 1 μM
Aprotinin 0.1 μM
E64 8 μM
Pepstatin 1.5 μM
Bestatin 162 μM
Chymostatin 33 μM

After clarification of the cell homogenate by centrifugation at 525 g for 10 min, the resulting supernatant was used as a source of esterase activity and was stored at −80° C. until required.

### Measurement of Ester Cleavage

Hydrolysis of esters to the corresponding carboxylic acids can be measured using the cell extract, prepared as above. To this effect cell extract (˜30 μg/total assay volume of 0.5 ml) was incubated at 37° C. in a Tris-HCl 25 mM, 125 mM NaCl buffer, pH 7.5 at 25° C. At zero time the ester (substrate) was then added at a final concentration of 2.5 μM and the samples were incubated at 37° C. for the appropriate time (usually 0 or 80 min). Reactions were stopped by the addition of 3× volumes of acetonitrile. For zero time samples the acetonitrile was added prior to the ester compound. After centrifugation at 12000 g for 5 min, samples were analysed for the ester and its corresponding carboxylic acid at room temperature by LCMS (Sciex API 3000, HP1100 binary pump, CTC PAL). Chromatography was based on an AcCN (75×2.1 mm) column and a mobile phase of 5-95% acetonitrile in water/0.1% formic acid.

The table below presents data showing that several amino acid ester motifs, conjugated to various intracellular enzyme inhibitors by several different linker chemistries are all hydrolysed by intracellular carboxyesterases to the corresponding acid.

| | | | | Preparation of |
|---|---|---|---|---|
| | | | Hydrolysis Rate Range | amino ester |
| Structure of amino acid ester conjugate | R | Linker | U937Cells (pg/mL/min) | conjugate |
| 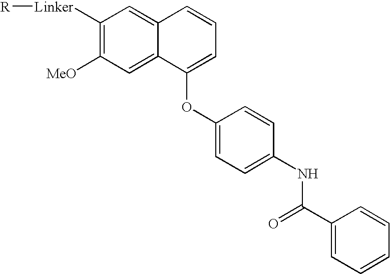 | 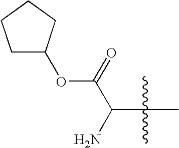 | —CH2CH2O— | 100-1000 | WO2006117552 |
| 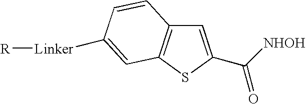 | 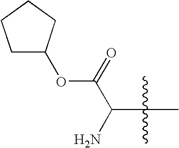 | 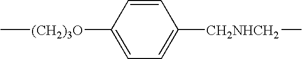 | 1000-50000 | WO2006117548 |
| 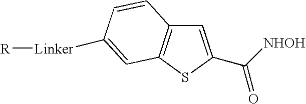 | 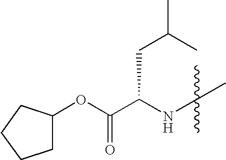 | 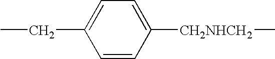 | >50000 | WO2006117549 |
| 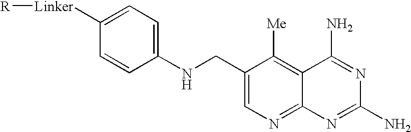 | 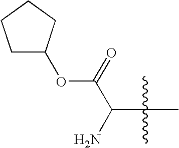 | —CH2CH2O— | >50000 | WO2006117567 |
| 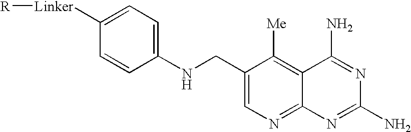 | 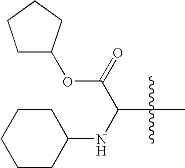 | —CH2CH2O— | 1000-50000 | WO2006117567 |
| 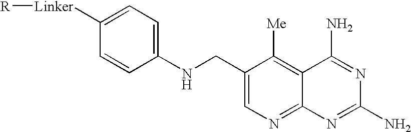 | 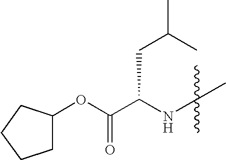 | —CH2— | 1000-50000 | WO2006117567 |
| 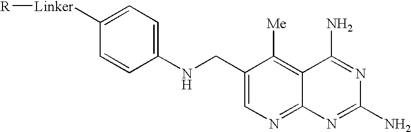 | 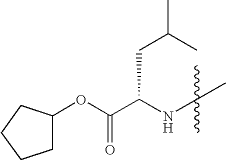 | —CO— | >50000 | WO2006117567 |
| 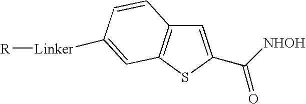 | 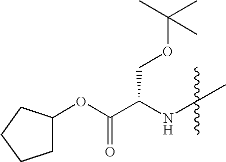 | 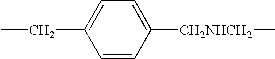 | >50000 | WO2006117549 |
| 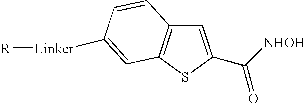 | 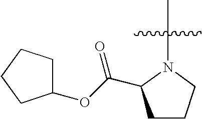 | 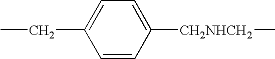 | >50000 | WO2006117549 |

### PLK1 Enzyme Assay

The ability of compounds to inhibit PLK-1 kinase activity was measured in an assay performed by Invitrogen (Paisley, UK). The Z′-LYTE™ biochemical assay employs a fluorescence-based, coupled-enzyme format and is based on the differential sensitivity of phosphorylated and non-phosphorylated peptides to proteolytic cleavage. The peptide substrate is labelled with two fluorophores—one at each end—that make up a FRET pair. In the primary reaction, the kinase transfers the gamma-phosphate of ATP to a single serine or threonine residue in a synthetic FRET-peptide. In the secondary reaction, a site-specific protease recognizes and cleaves non-phosphorylated FRET-peptides. Phosphorylation of FRET-peptides suppresses cleavage by the Development Reagent. Cleavage disrupts FRET between the donor (i.e., coumarin) and acceptor (i.e., fluorescein) fluorophores on the FRET-peptide, whereas uncleaved, phosphorylated FRET-peptides maintain FRET. A radiometric method, which calculates the ratio (the Emission Ratio) of donor emission to acceptor emission after excitation of the donor fluorophore at 400 nm, is used to quantitate reaction progress.

The final 10 μl Kinase Reaction consists of 2.8-25.3 ng PLK1, 2 μM Ser/Thr 16 Peptide substrate and ATP in 50 mM HEPES pH 7.5, 0.01% BRIJ-35, 10 mM MgCl2, 1 mM EGTA. The assay is performed at an ATP concentration at, or close to, the Km. After the 60 minute Kinase Reaction incubation at room temperature, 5 μl of a 1:8 dilution of Development Reagent is added. The assay plate is incubated for a further 60 minutes at room temperature and read on a fluorescence plate reader.

Duplicate data points are generated from a ⅓ log dilution series of a stock solution of test compound in DMSO. Nine dilutions steps are made from a top concentration of 10 μM, and a “no compound” blank is included. Data is collected and analysed using XLfit software from IDBS. The dose response curve is curve fitted to model number 205 (sigmoidal dose-response model). From the curve generated, the concentration giving 50% inhibition is determined and reported.

IC₅₀ results were allocated to one of 3 ranges as follows:

### Range A: IC50<100 nM,

Range B: IC50 from 100 nM to 500 nM;

### and Range C: IC50>500 nM.

NT=Not tested

The results obtained for compounds of the Examples herein are given in the Table below.

### Cell Inhibition Assay

Cells were seeded in 96W tissue culture plates (1 well=30 mm²) at a density of 500 cells per well in 50 μl of the appropriate culture medium (see below). 24 hrs later 50 μl of the compound prepared in the same medium was added as 4 fold dilutions to give final concentrations in the range 0.15 nM-2500 nM (n=6 for each concentration). The plates were then incubated at 37° C., 5% CO₂ for 120 hrs. Cell proliferation was assessed using WST-1 (a metabolic indicator dye, Roche Cat no. 1 644 807) according to the manufacturers instructions. The results were calculated as percentage of vehicle response and IC50 values represent the concentration of compound that inhibited the vehicle response by 50%.

HCT-116 Culture Medium—Dulbeccos MEM (Sigma D6546) plus 10% heat inactivated fetal calf serum (Hyclone SH30071 Thermo Fischer Scientific) containing 2 mM Glutamine (Sigma cat no G-7513) and 50 U/ml Penicillin and Streptomycin Sulphate (Sigma Cat no P-0781).

IC₅₀ results were allocated to one of 3 ranges as follows:

### Range A: IC50<100 nM,

Range B: IC50 from 100 nM to 500 nM;

### and Range C: IC50>500 nM.

NT=Not tested

The results obtained for compounds of the Examples herein are given in the Table below.

| | Inhibitor Activity | Inhibitor Activity |
|---|---|---|
| Example | vs | vs |
| Number | PLK1 | HCT 116 cell line |
| 1 | A | A |
| 2 | A | A |
| 3 | A | A |
| 4 | A | A |
| 5 | B | C |
| 6 | A | A |
| 7 | B | A |
| 8 | A | A |
| 9 | A | A |
| 10 | B | A |
| 11 | A | A |
| 12 | B | A |
| 13 | B | B |
| 14 | B | C |
| 15 | C | C |
| 16 | B | C |
| 17 | A | A |
| 18 | A | NT |
| 19 | A | NT |
| 20 | A | NT |
| 21 | A | NT |
| 22 | A | NT |
| 23 | A | NT |
| 24 | A | NT |
| 25 | A | NT |
| 26 | A | NT |
| 27 | A | NT |
| 28 | A | NT |
| 29 | A | NT |
| 30 | A | NT |
| 31 | A | NT |

1. A compound of formula (I), or a salt, N-oxide, hydrate or solvate thereof: 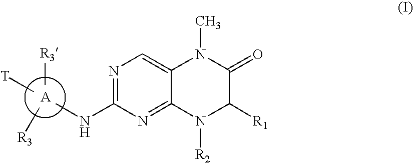
wherein
R₁ is hydrogen, or a (C₁-C₆)alkyl, (C₂-C₆)alkenyl, (C₂-C₆)alkynyl or (C₃-C₆)cycloalkyl group;
R₂ is hydrogen, or an optionally substituted (C₁-C₆)alkyl, (C₂-C₆)alkenyl, (C₂-C₆)alkynyl or (C₃-C₆)cycloalkyl group;
R₃ and R₃′ are independently selected from hydrogen, —CN, hydroxyl, halogen, optionally substituted (C₁-C₆)alkyl, (C₂-C₆)alkenyl, (C₂-C₆)alkynyl or (C₃-C₆)cycloalkyl, —NR₆R₇ or C₁-C₄ alkoxy, wherein R₆ and R₇ are independently hydrogen or optionally substituted (C₁-C₆)alkyl;
ring A is an optionally substituted mono- or bi-cyclic carbocyclic or heterocyclic ring or ring system having up to 12 ring atoms;
T is a radical of formula (II)
R₄R₅CH—NH—Y-L¹-X¹— (II)
wherein
R₄ is a carboxylic acid group (—COOH), or an ester group which is hydrolysable by one or more intracellular esterase enzymes to a carboxylic acid group;
R₅ is the side chain of a natural or non-natural alpha amino acid;
Y is a bond, —C(═O)—, —S(═O)₂—, —C(═O)O—, —C(═O)NR₆—, —C(═S)—NR₆, —C(═NH)—NR₆ or —S(═O)₂NR₆— wherein R₆ is independently hydrogen or optionally substituted (C₁-C₆)alkyl;
L¹ is a divalent radical of formula -(Alk¹)ₘ(Q¹)ₙ(Alk²)ₚ— wherein
m, n and p are independently 0 or 1,
Q¹ is (i) an optionally substituted divalent mono- or bicyclic carbocyclic or heterocyclic radical having 5-13 ring members, or (ii), in the case where p is 0, a divalent radical of formula -Q²-X²⁻ wherein X² is —O—, —S— or NR^{A}— wherein R^{A} is hydrogen or optionally substituted C₁-C₃ alkyl, and Q² is an optionally substituted divalent mono- or bicyclic carbocyclic or heterocyclic radical having 5-13 ring members,
Alk¹ and Alk² independently represent optionally substituted divalent (C₃-C₆)cycloalkyl radicals, or optionally substituted straight or branched, (C₁-C₆)alkylene, (C₂-C₆)alkenylene, or (C₂-C₆)alkynylene radicals which may optionally contain or terminate in an ether (—O—), thioether (—S—) or amino (—NR^{A}—) link wherein R^{A} is hydrogen or optionally substituted (C₁-C₃)alkyl;
X¹ represents a bond, —C(═O)—; or —S(═O)₂—; —NR₆C(═O)—, —C(═O)NR₆—, —NR₆C(═O)—NR₇—, —NR₆S(═O)₂—, or —S(═O)₂NR₆— wherein R₆ and R₇ are independently hydrogen or optionally substituted (C₁-C₆)alkyl.

2. A compound as claimed in claim 1 wherein R₁ is ethyl.

3. A compound as claimed in claim 1 wherein R₂ is cyclopentyl.

4. A compound as claimed in claim 1 wherein ring A is a phenyl ring.

5. A compound as claimed in claim 1 having formula (IA): 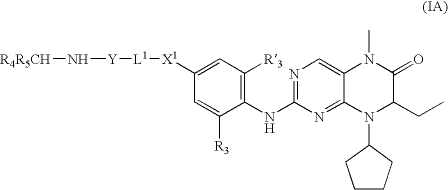
wherein R₃ is methoxy, fluoro or chloro, R₃′ is hydrogen, fluoro or chloro, and the remaining variables are as defined in claim 1.

6. A compound as claimed in claim 1 wherein R₄ is of formula
—(C═O)OR₁₀ wherein R₁₀ is R₁₁R₁₂R₁₃C— wherein
(i) R₁₁ is hydrogen or optionally substituted (C₁-C₃)alkyl-(Z¹)ₐ-[(C₁-C₃)alkyl]_{b}-, (C₂-C₃)alkenyl-(Z¹)ₐ-[(C₁-C₃)alkyl]_{b}- or phenyl-(Z¹)ₐ-[(C₁-C₃)alkyl]_{b}-, wherein a and b are independently 0 or 1 and Z¹ is —O—, —S—, or —NR₁₄— wherein R₁₄ is hydrogen or (C₁-C₃)alkyl; and R₁₂ and R₁₃ are independently hydrogen or (C₁-C₃)alkyl-;
(ii) R₁₁ is hydrogen or optionally substituted R₁₅R₁₆N—(C₁-C₃)alkyl- wherein R₁₅ is hydrogen, (C₁-C₃)alkyl or phenyl, and R₁₆ is hydrogen or (C₁-C₃)alkyl; or R₁₅ and R₁₆ together with the nitrogen to which they are attached form an optionally substituted monocyclic heterocyclic ring of 5- or 6-ring atoms or bicyclic heterocyclic ring system of 8 to 10 ring atoms, and R₁₂ and R₁₃ are independently hydrogen or (C₁-C₃)alkyl-; or
(iii) R₁₁ and R₁₂ taken together with the carbon to which they are attached form an optionally substituted monocyclic carbocyclic ring of from 3 to 7 ring atoms or bicyclic carbocyclic ring system of 8 to 10 ring atoms, and R₁₃ is hydrogen.

7. A compound as claimed in claim 1 wherein R₄ is a methyl, ethyl, n- or iso-propyl, n-, sec- or tert-butyl, cyclohexyl, allyl, phenyl, benzyl, 2-, 3- or 4-pyridylmethyl, N-methylpiperidin-4-yl, tetrahydrofuran-3-yl, methoxyethyl, indanyl, norbonyl, dimethylaminoethyl, or morpholinoethyl ester group.

8. A compound as claimed in claim 1 wherein R₄ is a cyclopentyl ester group.

9. A compound as claimed in claim 1 wherein R₅ is phenyl, benzyl, iso-butyl, cyclohexyl or t-butoxymethyl.

10. A compound as claimed in claim 1 wherein the radical —Y-L¹-X¹—, Y is
—C(═O)—, —C(═O)O— or —C(═O)NH—; X¹ is —NHC(═O)—; and L¹ has formula (IIIA), (IIB) or (IIC): 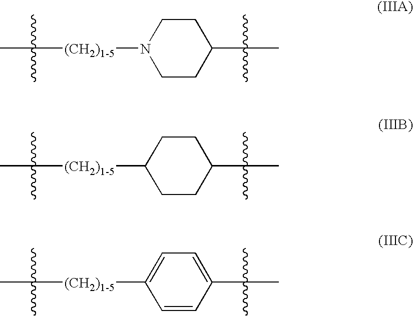
wherein the left hand valency is satisfied by Y and the right hand valency is satisfied by X¹.

11. A compound as claimed in claim 1 having formula (IB): 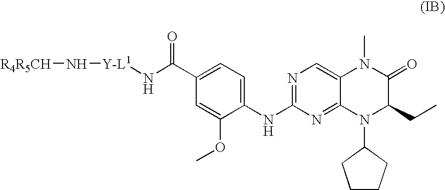
wherein R₄ is a methyl, ethyl, n- or iso-propyl, n-, sec- or tert-butyl, cyclohexyl, ally, phenyl, benzyl, 2-, 3- or 4-pyridylmethyl, N-methylpiperidin-4-yl, tetrahydrofuran-3-yl, methoxyethyl, indanyl, norbonyl, dimethylaminoethyl or morpholinoethyl ester group; and R₅ is phenyl, benzyl, iso-butyl, cyclohexyl or t-butoxymethyl; and Y and L¹ are as defined in claim 1.

12. A compound as claimed in claim 11 wherein Y is —C(═O)—, —C(═O)O— or —C(═O)NH—X¹ is —NHC(═O)—; and L¹ has formula (IIIA), (IIB) or (IIC): 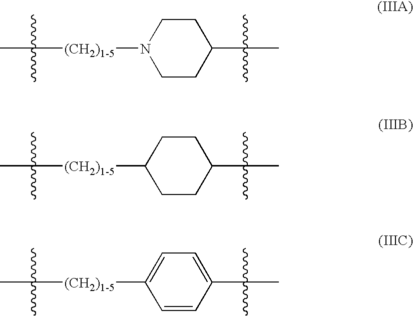
wherein the left hand valency is satisfied by Y and the right hand valency is satisfied by X¹.

13. A compound as claimed in claim 1, selected from the group consisting of:
Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}butanoyl)-L-leucinate,
Cyclopentyl N-(5-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}pentanoyl)-L-leucinate
Cyclopentyl N-[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethyl)carbamoyl]-L-leucinate,
Cyclopentyl N-[(2-{4-[(4-{[(7R)-8-cyclopentyl-7-ethyl-5-methyl-6-oxo-5,6,7,8-tetrahydropteridin-2-yl]amino}-3-methoxybenzoyl)amino]piperidin-1-yl}ethoxy)carbonyl]-L-leucinate,
Cyclopentyl N-(4-{4-[(4-{[(7R)-8-cyclopentyl-7-ethyl-5-methyl-6-oxo-5,6,7,8-tetrahydropteridin-2-yl]amino}-3-methoxybenzoyl)amino]phenyl}butanoyl)-L-leucinate,
Cyclopentyl N-(3-{cis-4-[(4-{[(7R)-8-cyclopentyl-7-ethyl-5-methyl-6-oxo-5,6,7,8-tetrahydropteridin-2-yl]amino}-3-methoxybenzoyl)amino]cyclohexyl}propanoyl)-L-leucinate,
And salts, hydrates and solvates thereof.

14. A pharmaceutical composition comprising a compound as claimed in claim 1, together with a pharmaceutically acceptable carrier.

15. A composition comprising an amount of a compound as claimed in claim 1 effective for inhibition of PLK1 activity in vitro or in vivo.

16. A method of treatment of conditions mediated by PLK1 activity, which comprises administering to a subject suffering such disease an effective amount of a compound as claimed in claim 1.

17. The method as claimed in claim 16 for treatment of cell proliferative diseases.

18. The method as claimed in claim 16 for treatment of solid tumours.

19. The method as claimed in claim 16 for treatment of haemato-oncological tumours.
